(19) United States
(12) Patent Application Publication
(10) Pub. No.: US 2004/0067927 A1
(43) Pub. Date: Apr. 8, 2004

(54) Substituted alkyldiamines

(76) Inventors: Christoph Boss, Allschwil (CH); Walter Fischli, Allschwil (CH); Solange Meyer, Schlierbach (FR); Sylvia Richard-Bildstein, Dietwiller (FR); Thomas Weller, Binningen (CH)

Correspondence Address: JONES DAY, 222 EAST 41ST STREET, NEW YORK, NY, 10017, US

(21) Appl. No.: 10/416,363

(22) PCT Filed: Oct. 31, 2001
(86) PCT No.: PCT/EP01/12617

(51) Int. Cl. A61K 31/397; C07C 271/00; A61K 31/55; C07D 213/26; A61K 31/40; A61K 31/44

(52) U.S. Cl. 514/210.01; 514/357; 514/408; 514/217.12; 514/478; 514/595; 540/609; 548/950; 548/571; 546/329; 560/24

(57) ABSTRACT

The invention relates to novel compounds which are substituted alkyldiamino derivatives of formula (I). The invention also concerns related aspects including processes for the preparation of the compounds, pharmaceutical compositions containing one or more compounds of formula (I) and especially their use as inhibitors of the plasmodium falciparum protease plasmepsin II or related aspartic proteases.

The invention relates to novel compounds which are substituted alkyldiamino derivatives of the general formula I. The invention also concerns related aspects including processes for the preparation of the compounds, pharmaceutical compositions containing one or more compounds of general formula I and especially their use as inhibitors of the plasmodium falciparum protease plasmepsin II or related aspartic proteases.

### BACKGROUND OF THE INVENTION

Malaria is one of the most serious and complex health problems affecting humanity in the 21^{st} century. The disease affects about 300 million people worldwide, killing 1 to 1.5 million people every year. Malaria is an infectious disease caused by four species of the protozoan parasite Plasmodium, P. falciparum being the most severe of the four. All attempts to develop vaccines against P. falciparum have failed so far. Therefore, therapies and preventive measures against malaria are confined to drugs. However, resistance to many of the currently available antimalarial drugs is spreading rapidly and new drugs are needed.

P. falciparum enters the human body by way of bites of the female anophelino mosquito. The plasmodium parasite initially populates the liver, and during later stages of the infectious cycle reproduces in red blood cells. During this stage, the parasite degrades hemoglobin and uses the degradation products as nutrients for growth [1]. Hemoglobin degradation is mediated by serine proteases and aspartic proteases. Aspartic proteases have been shown to be indispensable to parasite growth. A non-selective inhibitor of aspartic proteases, Pepstatin, inhibits the growth of P. falciparum in red blood cells in vitro. The same results have been obtained with analogs of pepstatin [2], [3]. These results show that inhibition of parasite aspartic proteases interferes with the life cycle of P. falciparum. Consequently, aspartic proteases are targets for antimalarial drug development.

The present invention relates to the identification of novel low molecular weight, non-peptidic inhibitors of the plasmodium falciparum protease plasmepsin II or other related aspartic proteases to treat and/or prevent malaria.

The compounds of general formula I were tested against plasmepsin II, HIV-protease, human cathepsin D, human cathepsin E and human renin in order to determine their biological activity and their selectivity profile.

### In Vitro Assays

The fluorescence resonance energy transfer (FRET) assay for HIV, plasmepsin II, human cathepsin D and human cathepsin E.

The assay conditions were selected according, to reports in the literature [4-7]. The FRET assay was performed in white polysorp plates (Fluoronunc, cat n° 437842 A). The assay buffer consisted of 50 mM Na acetate pH 5, 12,5% glycerol, 0.1% BSA+392 mM NaCl (for HIV-protease). The incubates per well were composed of:

160 μl buffer

10 μl inhibitor (in DMSO)

10 μl of the corresponding substrate in DMSO (see table A) to a final concentration of 1 μM

20 μl of enzyme to a final amount of x ng per assay tube (x=10 ng/assay tube plasmepsin II, x=100 ng/assay tube HIV-protease, x=10 ng/assay tube human cathepsin E and x=20 ng/assay tube human cathepsin D)

The reactions were initiated by addition of the enzyme. The assay was incubated at 37° C. for 30 min (for human cathepsin E), 40 min (for plasmepsin II and HIV-protease) or 120 min (for human cathepsin D). The reactions were stopped by adding 10% (v/v) of a 1 M solution of Tris-base. Product-accumulation was monitored by measuring the fluorescence at 460 nm.

Auto-fluorescence of all the test substances is determined in assay buffer in the absence of substrate and enzyme and this value was subtracted from the final signal.

TABLE A
Summary of the conditions used for the aspartyl proteases
fluorescent assays. (at = assay tube)

| | substrate | | enzyme | | | |
|---|---|---|---|---|---|---|
| | | substrate | concentration | | | Incubation |
| Aspartyl | | concentration | ng/at | | | time |
| protease | sequence | μM | (nM) | Buffer | pH | minutes |
| HIV | Dabcyl-Abu-SQNY:PIVN-EDANS | 1 | 100 | 50 mM Na acetate; | 5 | 40 |
| | | | (22.5) | 12.5% glycerol; | | |
| | | | | 0.1% BSA | | |
| | | | | 392 mM NaCl | | |
| Plasmepsin II | Dabcyl-ERNleF:LSFP-EDANS | 1 | 10 | 50 mM Na acetate; | 5 | 40 |
| | | | (1.25) | 12.5% glycerol; | | |
| | | | | 0.1% BSA | | |
| h Cathepsin D | Dabcyl-ERNleF:LSFP-EDANS | 1 | 20 | 50 mM Na acetate; | 5 | 120 |
| | | | (2.5) | 12.5% glycerol; | | |
| | | | | 0.1% BSA | | |
| h Cathepsin E | Dabcyl-ERNleF:LSFP-EDANS | 1 | 10 | 50 mM Na acetate; | 6 | 30 |
| | | | (1.25) | 12.5% glycerol; | | |
| | | | | 0.1% BSA | | |

### Enzymatic in Vitro Assay for Renin

The enzymatic in vitro assay was performed in polypropylene plates (Nunc, Cat No 4-42587A). The assay buffer consisted of 100 mM sodium phosphate, pH 7.4, including 0.1% BSA. The incubates were composed of 190 μL per well of an enzyme mix and 10 μL of renin inhibitors in DMSO. The enzyme mix was premixed at 4° C. and composed as follows:

human recombinant renin (0.16 ng/mL)

synthetic human tetradecapeptide renin substrate (0.5 μM)

hydroxyquinoline sulfate (0.1 mM)

The mixtures were then incubated at 37° C. for 3 h.

To determine the enzymatic activity and its inhibition, the accumulated Angiotensin I was detected by an enzyme immunoassay (EIA). 10 μL of the incubates or standards were transferred to immuno plates which were previously coated with a covalent complex of Angiotensin I and bovine serum albumin (Ang I-BSA). 190 μL of Angiotensin I-antibodies were added and a primary incubation made at 4° C. over night. The plates were washed 3 times and then incubated for one hour at room temperature with a biotinylated anti-rabbit antibody. Thereafter, the plates were washed and incubated at room temperature for 30 min with a streptavidin-peroxidase complex. After washing the plates, the peroxidase substrate ABTS (2,2′-Azino-di-(3-ethyl-benzthiazolinsulfonate), was added and the plates incubated for 10-30 min at room temperature. After stopping the reaction with 0.1 M citric acid pH 4.3 the plate is evaluated in a microplate reader at 405 nm.

TABLE 1
IC₅₀ values (nM) for selected compounds on plasmepsin II:

| Example Nr: | IC50 (nM) on plasmepsin II |
|---|---|
| Example 1 | 115 |
| Example 21 | 469 |
| Example 22 | 858 |
| Example 23 | 252 |
| Example 25 | 596 |
| Example 20 | 846 |
| Example 38 | 325 |
| Example 51 | 691 |
| Example 52 | 834 |
| Example 53 | 125 |
| Example 54 | 312 |
| Example 56 | 659 |
| Example 57 | 351 |
| Example 58 | 754 |
| Example 59 | 380 |
| Example 60 | 198 |
| Example 61 | 57 |
| Example 68 | 714 |
| Example 69 | 8230 |

### References

1. Goldberg, D. E., Slater, A. F., Beavis, R., Chait, B., Cerami, A., Henderson, G. B., Hemoglobin degradation in the human malaria pathogen Plasmodium falciparum: a catabolic pathway initiated by a specific aspartic protease; J. Exp. Med., 1991, 173, 961-969.

2. Francis, S. E., Gluzman, I. Y., Oksman, A., Knickerbocker, A., Mueller, R., Bryant, M. L., Sherman, D. R., Russell, D. G., Goldberg, D. E., Molecular characterization and inhibition of a Plasmodium falciparum aspartic hemoglobinase; Embo. J., 1994, 13, 306-317.

3. Moon, R. P., Tyas, L., Certa, U., Rupp, K., Bur, D., Jaquet, H., Matile, H., Loetscher, H., Grueninger-Leitch, F., Kay, J., Dunn, B. M., Berry, C., Ridley, R. G., Expression and characterization of plasmepsin I from Plasmodium falciparum, Eur. J. Biochem., 1997, 244, 552-560.

4. Carroll, C. D., Johnson, T. O., Tao, S., Lauri, G., Orlowski, M., Gluzman, I. Y., Goldberg, D. E., Dolle, R. E., (1998). “Evaluation of a structure-based statine cyclic diamino amide encoded combinatorial library against plasmepsin II and cathepsin D”. Bioorg Med Chem Lett; 8(22), 3203-3206.

5. Peranteau, A. G., Kuzmic, P., Angell, Y., Garcia-Echeverria, C., Rich, D. H., (1995). “Increase in fluorescence upon the hydrolysis of tyrosine peptides: application to proteinase assays”. Anal Biochem; 227(1):242-245.

6. Gulnik, S. V., Suvorov, L. I., Majer, P., Collins, J., Kane, B. P., Johnson, D. G., Erickson, J. W., (1997). “Design of sensitive fluorogenic substrates for human cathepsin D”. FEBS Lett; 413(2), 379-384.

7. Robinson, P. S., Lees, W. E., Kay, J., Cook, N. D., (1992). “Kinetic parameters for the generation of endothelins-1, -2 and -3 by human cathepsin E”. Biochem J; 284 (Pt 2): 407-409.

8. J. March, Advanced Organic Chemistry, pp 918-919, and refs. cited therein; 4^{th}Ed., John Wiley & Sons, 1992.

9. A. Kubo, N. Saito, N. Kawakami, Y. Matsuyama, T. Miwa, Synthesis, 1987, 824-827.

10. R. K. Castellano, D. M. Rudkevich, J. Rebek, Jr., J. Am. Chem. Soc., 1996, 118, 10002-10003.

11. U. Schöllkopf, Pure Appl. Chem., 1983, 55, 1799-1806 and refs. cited therein; U. Schïllkopf, Top. Curr. Chem., 1983, 109, 65-84 and refs. cited therein; T. Wirth, Angew. Chem. Int. Ed. Engl., 1997, 36, 225-227 and refs. cited therein.

12. T. W. Greene, P. G. M. Wutts, Protective groups in organic synthesis; Wiley-Interscience, 1991.

13. P. J. Kocienski, Protecting Groups, Thieme, 1994.

14. J. A. Radding, Development of Anti-Malarial Inhibitors of Hemoglobinases, Annual Reports in Medicinal Chemistry, 34, 1999, 159-168.

15. D. F. Wirth, Malaria: A Third World Disease in Need of First World Drug Development, Annual Reports in Medicinal Chemistry, 34, 1999, 349-358.

The present invention relates to novel, low molecular weight organic compounds, which are substituted dialkylamines of the general formula I:

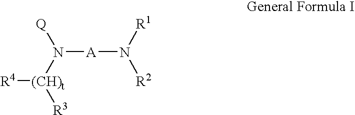

wherein

Q represents —SO₂—R⁵; —CO—R⁵; —CO—NH—R⁵; —CO—N(R⁵)(R⁶); —CO—OR⁵; —(CH₂)ₚ—R⁵; —(CH₂)ₚ—CH(R⁵)(R⁶);

R¹ and R² represent propyl; butyl; pentyl; hexyl; ω-hydroxy-propyl; ω-hydroxy-butyl; ω-hydroxy-pentyl; ω-hydroxy-hexyl; lower alkoxy-propyl; lower alkoxy-butyl; lower alkoxy-pentyl; lower alkoxy-hexyl; aryl-lower alkyl; cycloalkyl; cycloalkyl-lower alkyl; heterocyclyl; and can be the same or different; or R¹ and R² and the nitrogen atom together can represent a ring such as azetidin; azepan;

R³ represents lower alkyl; lower alkenyl; aryl; heteroaryl; cycloalkyl; heterocyclyl; aryl-lower alkyl; heteroaryl-lower alkyl; cycloalkyl-lower alkyl; heterocyclyl-lower alkyl; aryl-lower alkenyl; heteroaryl-lower alkenyl; cycloalkyl-lower alkenyl; heterocyclyl-lower alkenyl;

R⁴ represents hydrogen; —CH₂—OR⁷; —CO—OR⁷; lower alkyl;

R⁵ and R⁶ represent lower alkyl; lower alkenyl; aryl; heteroaryl; cycloalkyl; heterocyclyl; aryl-lower alkyl; heteroaryl-lower alkyl; cycloalkyl-lower alkyl; heterocyclyl-lower alkyl; aryl-lower alkenyl; heteroaryl-lower alkenyl; cycloalkyl-lower alkenyl; heterocyclyl-lower alkenyl;

R⁷ represents hydrogen, lower alkyl; cycloalkyl; aryl; cycloalkyl-lower alkyl; aryl-lower alkyl;

t represents the whole numbers 0 (zero) or 1 and in case t represents the whole number 0 (zero), R⁴ is absent;

p represents the whole numbers 0 (zero), 1 or 2;

A represents —CH₂)ₙ—;

n represents the whole numbers 2, 3, 4 or 5;

and pure enantiomers, mixtures of enantiomers, pure diastereomers, mixtures of diastereomers, diastereomeric racemates, mixtures of diastereomeric racemates and pharmaceutically acceptable salts thereof.

In the definitions of the general formula I—if not otherwise stated—the expression lower means straight and branched chain groups with one to seven carbon atoms, preferably 1 to 4 carbon atoms. Examples of lower alkyl groups are methyl, ethyl, n-propyl, iso-propyl, n-butyl, iso-butyl, sec.-butyl, tert.-butyl, pentyl, hexyl, heptyl. Examples of lower alkoxy groups are methoxy, ethoxy, propoxy, iso-butoxy, sec.-butoxy and tert.-butoxy etc. Lower alkylendioxy-groups as substituents of aromatic rings onto two adjacent carbon atoms are preferably methylene-dioxy and ethylene-dioxy. Lower alkylen-oxy groups as substituents of aromatic rings onto two adjacent carbon atoms are preferably ethylen-oxy and propylen-oxy. Examples of lower alkanoyl-groups are acetyl, propanoyl and butanoyl. Lower alkenylen means e.g. vinylen, propenylen and butenylen.

The expression cycloalkyl, alone or in combination, means a saturated cyclic hydrocarbon ring system with 3 to 6 carbon atoms, e.g. cyclopropyl, cyclobutyl, cyclopentyl and cyclohexyl which may be substituted with lower alkyl groups.

The expression heterocyclyl, alone or in combination, means saturated or unsaturated (but not aromatic) five-, six- or seven-membered rings containing one or two nitrogen, oxygen or sulfur atoms which may be the same or different and which rings may be substituted with lower alkyl, lower alkenyl, aryl; examples of such rings are morpholinyl, piperazinyl, tetrahydropyranyl, dihydropyranyl, 1,4-dioxanyl, pyrrolidinyl, tetrahydrofuranyl, dihydropyrrolyl, imidazolidinyl, dihydropyrazolyl, pyrazolidinyl etc. and substituted derivatives of such type rings with substituents as outlined hereinbefore.

The expression heteroaryl, alone or in combination, means six-membered aromatic rings containing one to four nitrogen atoms; benzofused six-membered aromatic rings containing one to three nitrogen atoms; five-membered aromatic rings containing one oxygen, one nitrogen or one sulfur atom; benzo-fused five-membred aromatic rings containing one oxygen, one nitrogen or one sulfur atom; five membered aromatic rings containing one oxygen and one nitrogen atom and benzo fused derivatives thereof; five membred aromatic rings containing a sulfur and nitrogen or oxygen atom and benzo fused derivatives thereof; five membered aromatic rings containing three nitrogen atoms and benzo fused derivatives thereof or the tetrazolyl ring; examples of such rings are furanyl, thienyl, pyrrolyl, pyridinyl, indolyl, quinolinyl, isoquinolinyl, dihydroquinolinyl, tetrahydroquinolinyl, tetrahydroisoquinolinyl, imidazolyl, triazinyl, thiazinyl, pyridazinyl, oxazolyl, and the like, whereby such ring systems may be mono-, di- or tri-substituted with aryl; aryloxy, aryl-lower alkoxy, lower alkyl; lower alkenyl; lower alkyl-carbonyl; amino; lower alkyl-amino; bis-(lower-alkylyamino; lower alkanoyl-amino; lower alkyl-sulfonamido; aryl-sulfonamido, heteroaryl-sulfonamido; lower alkyl-sulfono; aryl-sulfono; ω-amino-lower alkyl; halogen; hydroxy; carboxyl; lower alkoxy; vinyloxy; allyloxy; ω-hydroxy-lower alkyl; nitro; cyano; amidino; trifluoromethyl; lower alkyl-sulfonyl.

The expression aryl, alone or in combination, means six membered aromatic rings and condensed systems like naphthyl or indenyl, whereby such ring systems may be mono-, di- or tri-substituted with aryl, aryloxy, aryl-lower alkyloxy, lower alkyl, lower alkenylen, lower alkyl-carbonyl, aryl-carbonyl, amino, lower alkyl-amino, aryl-amino, bis-(lower-alkyl)-amino, lower alkanoyl-amino, lower alkyl-sulfonamido, aryl-sulfonamido, heteroaryl-sulfonamido, lower alkyl-sulfono, aryl-sulfono, ω-amino-lower alkyl, halogen, hydroxy, carboxyl, lower alkoxy, vinyloxy, allyloxy, ω-hydroxy-lower alkyl, ω-hydroxy-lower alkoxy, nitro, cyano, amidino, trifluoromethyl, lower alkyl-sulfonyl. In the case where the substituent on the aryl unit is another aryl unit, this second aryl unit may again be mono-, di- or tri-substituted with the substituents given as examples above.

It is understood that the substituents outlined relative to the expressions cycloalkyl, heterocyclyl, heteroaryl and aryl have been omitted in the definitions of the general formulae I to VI and in claims 1 to 6 for clarity reasons but the definitions in formulae I to VI and in claims 1 to 6 should be read as if they are included therein.

The expression pharmaceutically acceptable salts encompasses either salts with inorganic acids or organic acids like hydrochloric or hydrobromic acid; sulfuric acid, phosphoric acid, nitric acid, citric acid, formic acid, acetic acid, maleic acid, tartaric acid, methylsulfonic acid, p-toluolsulfonic acid and the like or in case the compound of formula I is acidic in nature with an inorganic base like an alkali or earth alkali base, e.g. sodium hydroxide, potassium hydroxide, calcium hydroxide.

The compounds of the general formula I can contain one or more asymmetric carbon atoms and may be prepared in form of optically pure enantiomers, mixtures of enantiomers, pure diastereomers, mixtures of diastereomers, diastereomeric racemates and mixtures of diastereomeric racemates.

The present invention encompasses all these forms. Mixtures may be separated in a manner known per se, i.e. by column chromatography, thin layer chromatography, HPLC or crystallization.

The compounds of the general formula I and their pharmaceutically acceptable salts may be used as therapeutics e.g. in form of pharmaceutical compositions. They may especially be used to in prevention or treatment of malaria. These compositions may be administered in enteral or oral form e.g. as tablets, dragees, gelatine capsules, emulsions, solutions or suspensions, in nasal form like sprays or rectally in form of suppositories. These compounds may also be administered in intramuscular, parenteral or intraveneous form, e.g. in form of injectable solutions.

These pharmaceutical compositions may contain the compounds of formula I as well as their pharmaceutically acceptable salts in combination with inorganic and/or organic excipients which are usual in the pharmaceutical industry like lactose, maize or derivatives thereof, talcum, stearinic acid or salts of these materials.

For gelatine capsules vegetable oils, waxes, fats, liquid or half-liquid polyols may be used. For the preparation of solutions and sirups e.g. water, polyols saccharose, glucose and related materials are used. Injectables are prepared by using e.g. water, polyols, alcohols, glycerin, vegetable oils, lecithin, liposomes and the like. Suppositories are prepared by using natural or hydrogenated oils, waxes, fatty acids (fats), liquid or half-liquid polyols.

The compositions may contain in addition preservatives, stability improving substances, viscosity improving or regulating substances, solubility improving substances, sweeteners, dyes, taste improving compounds, salts to change the osmotic pressure, buffer, anti-oxidants and related materials.

The compounds of formula I may also be used in combination with one or more other therapeutically useful substances e. g. with other antimalarials like quinolines (quinine, chloroquine, amodiaquine, mefloquine, primaquine, tafenoquine), peroxide antimalarials (artemisinin derivatives), pyrimethamine-sulfadoxine antimalarials (e.g. Fansidar), hydroxynaphtoquinones (e.g. atovaquone), acroline-type antimalarials (e. g. pyronaridine) and the like.

The dosage may vary within wide limits but should be adapted to the specific situation. In general the dosage given in oral form should daily be between about 3 mg and about 3 g, peferably between about 10 mg and about 1 g, especially preferred between 5 mg and 300 mg, per adult with a body weight of about 70 kg. The dosage should be administered preferably in 1 to 3 doses per day which are of equal weight. As usual, children should receive lower doses which are adapted to body weight and age.

Preferred compounds are compounds of the formula II

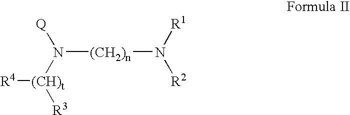

wherein

Q, t, R³ and R⁴ are as defined in general formula I above, R¹ and R² represent lower alkyl and n represents the whole numbers 2 or 3

and pure enantiomers, mixtures of enantiomers, pure diastereomers, mixtures of diastereomers, diastereomeric racemates, mixtures of diastereomeric racemates and pharmaceutically acceptable salts thereof.

Also preferred compounds are compounds of formula III

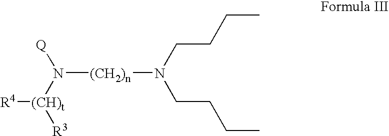

wherein

Q, t, R³ and R⁴ are as defined in general formula I above and n represents the whole numbers 2 or 3

and pure enantiomers, mixtures of enantiomers, pure diastereomers, mixtures of diastereomers, diastereomeric racemates, mixtures of diastereomeric racemates and pharmaceutically acceptable salts thereof.

Especially preferred are also compounds of the formula IV

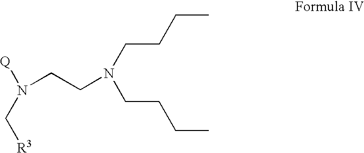

wherein

Q and R³ are as defined in general formula I above

and pure enantiomers, mixtures of enantiomers, pure diastereomers, mixtures of diastereomers, diastereomeric racemates, mixtures of diastereomeric racemates and pharmaceutically acceptable salts thereof.

Especially preferred are also compounds of the formula V

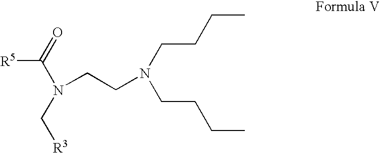

wherein R³ and R⁵ are as defined in general formula I above

and pure enantiomers, mixtures of enantiomers, pure diastereomers, mixtures of diastereomers, diastereomeric racemates, mixtures of diastereomeric racemates and pharmaceutically acceptable salts thereof.

Especially preferred are compounds of the formula VI

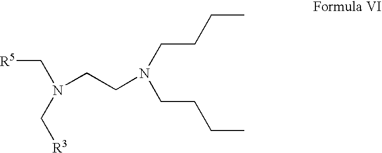

wherein R³ and R⁵ are as defined in general formula I above

and pure enantiomers, mixtures of enantiomers, pure diastereomers, mixtures of diastereomers, diastereomeric racemates, mixtures of diastereomeric racemates and pharmaceutically acceptable salts thereof.

Preferred compounds are:

N-(4-Benzyloxybenzyl)-N-(2-dibutylamino-ethyl)-4-pentylbenzamide;

N-Biphenyl-4-ylmethyl-N-(2-dibutylamino-ethyl)-4-pentylbenzamide;

N-(2-Dibutylaminoethyl)-N-[4′-(2-hydroxy-ethoxy)-biphenyl-4-ylmethyl]-4-pentylbenzamide;

N-(4-Benzo[1,3]dioxol-5-yl-benzyl)-N-(2-dibutyl-aminoethyl)-4-pentylbenzamide.

The compounds of the general formula I of the present invention may be prepared according to the general sequences of reactions outlined below, wherein R³, R⁴, R⁵, R⁶, R⁷, Q, A, t, n and p are as defined in general formula I above (for simplicity and clarity reasons, only parts of the synthetic possibilities which lead to compounds of formulae I to VI are described). For general methods of certain steps see also pages 16-18 and 20-21.

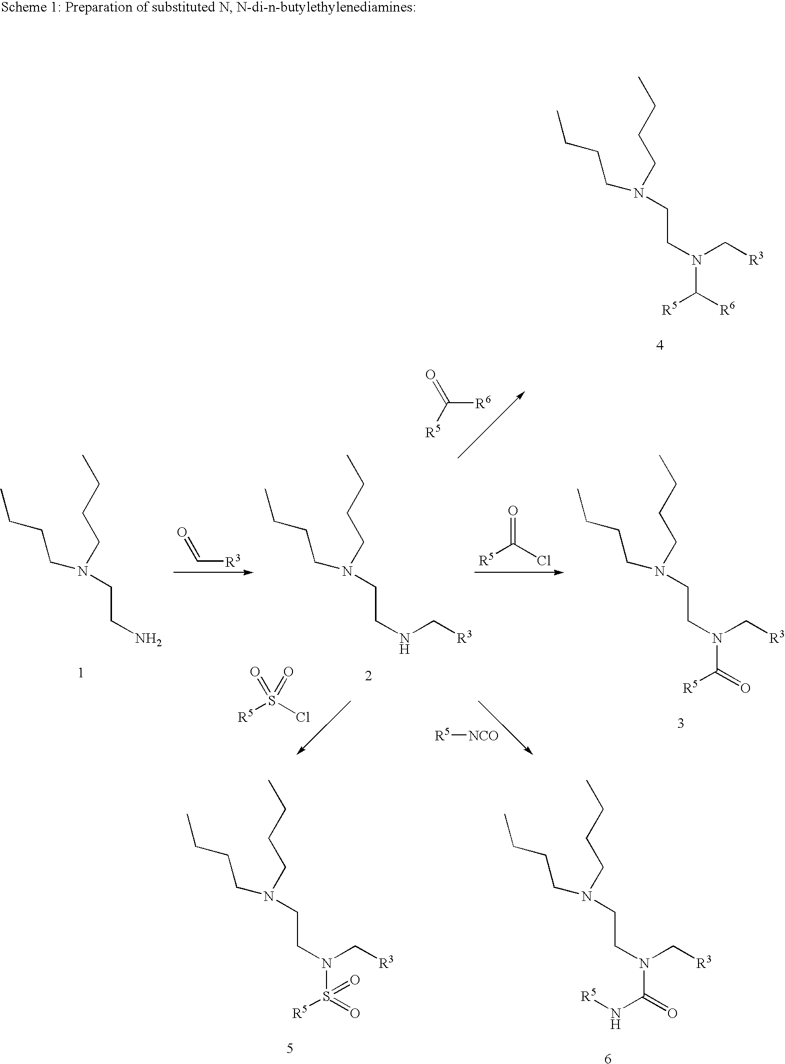

### Typical Procedure for the First Reductive Amination (Synthesis of Compound 2)

The amine (1) and the aldehyde {R³—CHO} (1.5 eq.) are mixed in anhydrous methanol and stirred for 6 h. The mixture is treated with sodium borohydride (1.5 eq.) and stirred for 2 h. Purified Amberlyst 15 or another suitable scavenger is added and the suspension is shaken for 12 h. The resin is separated by filtration and washed with methanol. The secondary amine 2 is removed from the resin by adding a 2M methanolic ammonia solution. After 30 min of shaking, the resin is filtered and washed with methanol. The filtrate is evaporated to yield the pure secondary amine 2.

If not comercially available, aryl- or heteroaryl substituted benzaldehydes can be prepared as follows:

The aldehyde {R³—CHO} may be obtained from commercially available formylbenzeneboronic acids and substituted bromo aryls or bromo heteroaryls via a Suzuki coupling as described in the literature or as described in the typical procedure D) below.

### Typical Procedure for the Acylation (Synthesis of Compound 3)

To a solution of the amine 2 in anhydrous ethyl acetate is added vacuum dried Amberlyst 21 or another suitable scavenger, followed by the addition of the carboxylic acid chloride {R⁵—(CO)—Cl} (1.5 eq.). After shaking the suspension for 2 hours, an aliquot water is added in order to hydrolyze the excess of carboxylic acid chloride and shaking is continued for 1 h. The resin is then removed by filtration, washed with ethyl acetate and the solution is evaporated to yield the pure amide 3.

The carboxylic acid chlorides {R⁵—(CO)—Cl} may be obtained in situ from the corresponding carboxylic acid as described in the literature (i. e.: Devos, A.; Rémion, J.; Frisque-Hesbain, A. -M.; Colens, A.; Ghosez, L., J. Chem. Soc., Chem. Commun. 1979, 1180).

The synthesis of the sulfonamide derivatives 5 from the amine 2 is performed in analogy to the above-described procedure.

The urea derivatives 6 are obtained by reaction of the amines 2 in dichloromethane with one equivalent of an isocyanate.

### Typical Procedure for the Second Reductive Amination (Synthesis of Compound 4)

The amine (1) and the ketone or aldehyde {R⁵R⁶CO} (1.5 eq.) are mixed in anhydrous dichloromethane and sodium triacetoxyborohydride (1.3 eq.) is added. After stirring the solution for 48 h, methanol is added and the reaction mixture is treated in the same manner as described for the amines 2.

Compounds of formula II, where R¹ and R² represent lower alkyl and n represents the whole number 2 or 3 are synthesized as described in scheme 1.

All chemical transformations can be performed according to well known standard methodologies as described in the literature or as described in the typical procedures above.

The following examples illustrate the invention but do not limit the scope thereof. All temperatures are stated in ° C.

### List of abbreviations:

| | |
|---|---|
| Boc or boc | tert.-butyloxycarbonyl |
| Cbz | benzyloxycarbonyl |
| DBU | 1,8-diazabicyclo[5.4.0]undec-7-ene(1,5—5) |
| DCM | dichloromethane |
| DMF | dimethylformamide |
| DMSO | dimethylsulfoxide |
| EtOAc | ethyl acetate |
| TEA | triethylamine |
| TFA | trifluoroacetic acid |
| THF | tetrahydrofuran |
| TLC | thin layer chromatography |

### General Procedures and Examples

The following compounds are prepared according to the procedures described for the synthesis of compounds encompassed by the general formulae hereinbefore. All compounds are caracterized by ¹H-NMR (300 MHz) and occasionnally by ¹³C-NMR (75 MHz) (Varian Oxford, 300 MHz; chemical shifts are given in ppm relative to the solvent used; multiplicities: s=singlet, d=doublet, t=triplet, m=multiplet), by LC-MS (Waters Micromass; ZMD-platform with ESI-probe with Alliance 2790 HT; column: 2×30 mm, Gromsil ODS4, 3 μM 120A; gradient: 0-100% acetonitrile in water, 6 min, with 0.05% formic acid, flow: 0.45 ml/min; tᵣ is given in minutes), by TLC (TLC-plates from Merck, silica gel 60 F₂₅₄) and occasionally by melting point.

### a) General Procedures

### Typical Procedure A) for the First Reductive Amination

The amine and the aldehyde (1.5 eq.) (which are used as starting materials, are known compounds or the synthesis (in case of the aldehydes) is described below in section c) in Referential Examples 1 to 6) are mixed in anhydrous methanol and stirred for 6 h. The mixture is then treated with sodium borohydride (1.5 eq.) and stirred for 2 h. Purified Amberlyst 15 or another suitable scavenger is added and the suspension is shaken for 12 h. The resin is then separated by filtration and washed with methanol. The secondary amine is removed from the resin by adding a 2M methanolic ammonia solution. After 30 min of shaking, the resin is filtered off and washed with methanol. The filtrate is evaporated to yield the pure secondary amine.

### Typical Procedure B) for the Acylation

To a solution of the amine in anhydrous ethyl acetate is added vacuum dried Amberlyst 21 or another suitable scavenger, followed by the addition of the carboxylic acid chloride (1.5 eq.) (which either are commercially available or prepared in situ from the corresponding carboxylic acids according to the literature). After shaking the suspension for 2 h, an aliquot of water is added in order to hydrolyze the excess of carboxylic acid chloride and shaking is continued for 1 h. The resin is then removed by filtration, washed with ethyl acetate and the solution is evaporated to yield the pure amide.

### Typical Procedure C) for the Second Reductive Amination

The amine and the aldehyde or the ketone (1.5 eq.) are mixed in anhydrous dichloromethane and sodium triacetoxyborohydride (1.3 eq.) is added. After stirring of the solution for 48 h, methanol is added and the reaction mixture is treated in the same manner as described in procedure A).

### Typical Procedure D) for the Suzuki Coupling

To a solution of the bromide in toluene, the boronic acid (1.1 eq.) dissolved in isopropanol is added followed by a 2M aqueous solution of potassium carbonate (5 eq.). The mixture is purged with nitrogen for 10 min and tetrakis(triphenylphosphine) palladium (0.03 eq.) is added. After heating under reflux for 6 h, water is added to the cooled reaction mixture and the product is extracted with ethyl acetate. The organic phase is washed with brine and dried over sodium sulfate. The solvent is evaporated to give the crude aldehyde, which is purified by flash chromatography (ethyl acetate/heptane gradient).

### b) EXAMPLES

### Example 1

According to typical procedure B), the secondary amine a), obtained via typical procedure A), is reacted with 4-n-pentylbenzoyl chloride to give

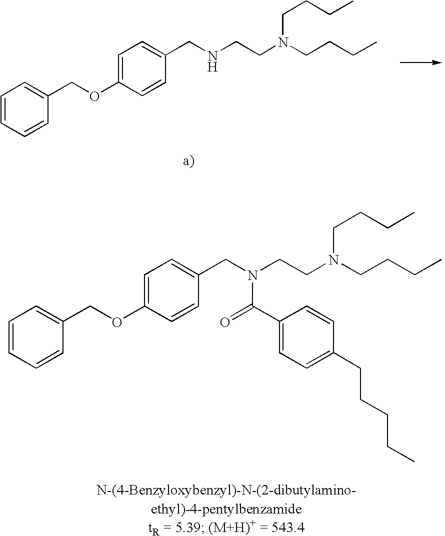

### Example 2

According to typical procedure B), the secondary amine a), obtained via typical procedure A), is reacted with 4-n-propylbenzoyl chloride to give

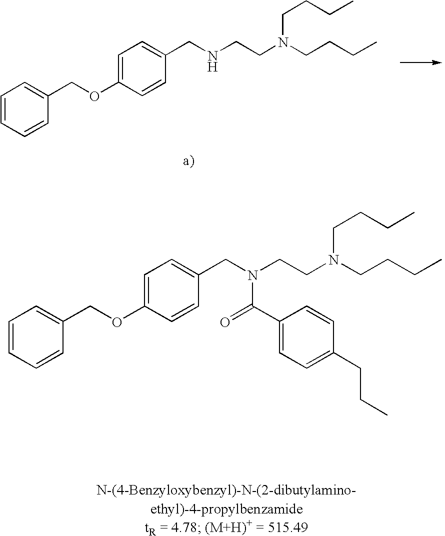

### Example 3

According to typical procedure B), the secondary amine b), obtained via typical procedure A), is reacted with 4-n-propylbenzoyl chloride to give

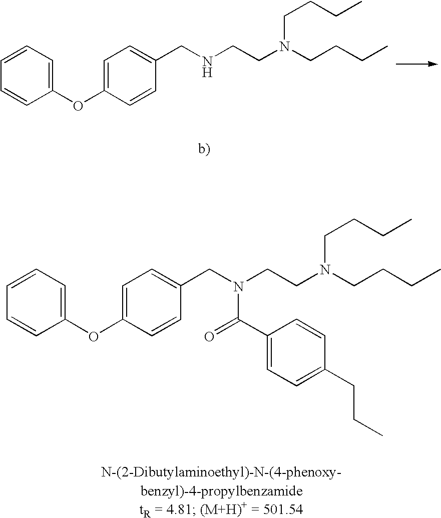

### Example 4

According to typical procedure B), the secondary amine c), obtained via typical procedure A), is reacted with 4-n-propylbenzoyl chloride to give

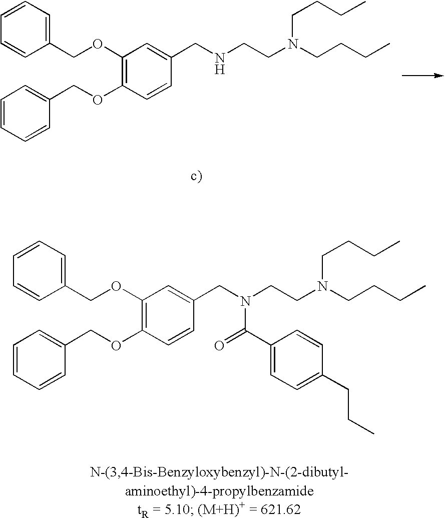

### Example 5

According to typical procedure B), the secondary amine d), obtained via typical procedure A), is reacted with 4-n-propylbenzoyl chloride to give

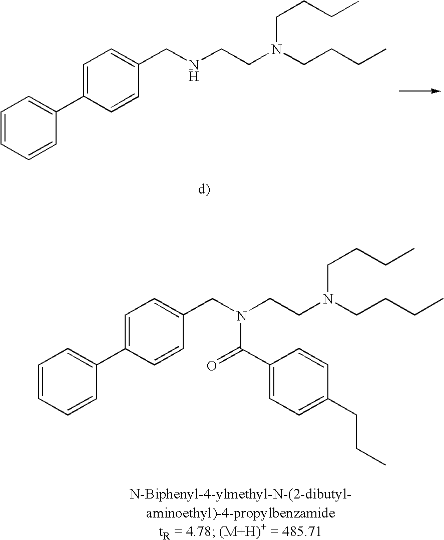

### Example 6

According to typical procedure B), the secondary amine e), obtained via typical procedure A), is reacted with 4-n-propylbenzoyl chloride to give

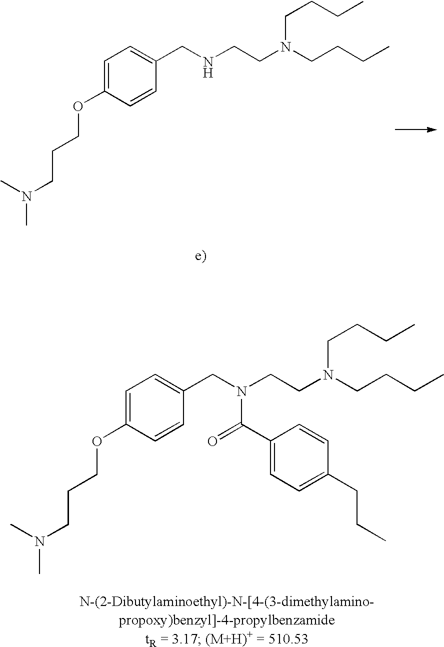

### Example 7

According to typical procedure B), the secondary amine f), obtained via typical procedure A), is reacted with 4-n-propylbenzoyl chloride to give

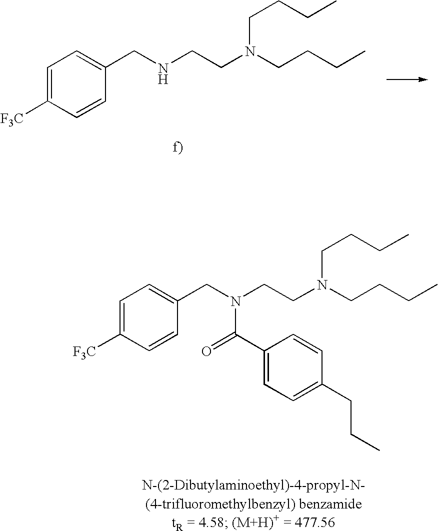

### Example 8

According to typical procedure B), the secondary amine g), obtained via typical procedure A), is reacted with 4-n-propylbenzoyl chloride to give

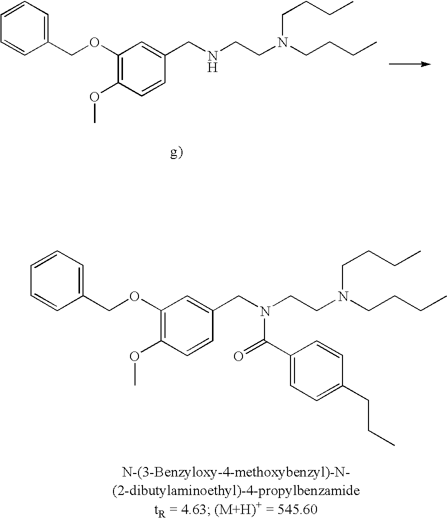

### Example 9

According to typical procedure B), the secondary amine h), obtained via typical procedure A), is reacted with 4-n-propylbenzoyl chloride to give

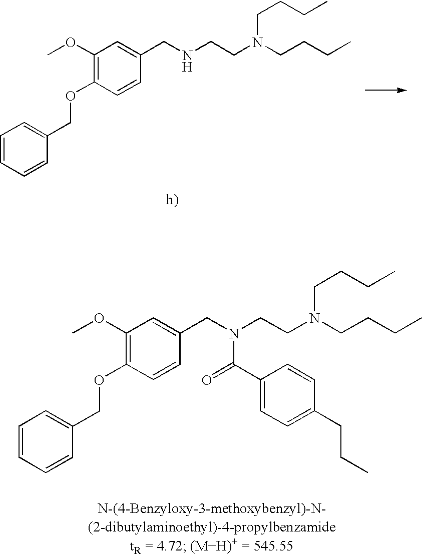

### Example 10

According to typical procedure B), the secondary amine a), obtained via typical procedure A), is reacted with 4-n-butylbenzoyl chloride to give

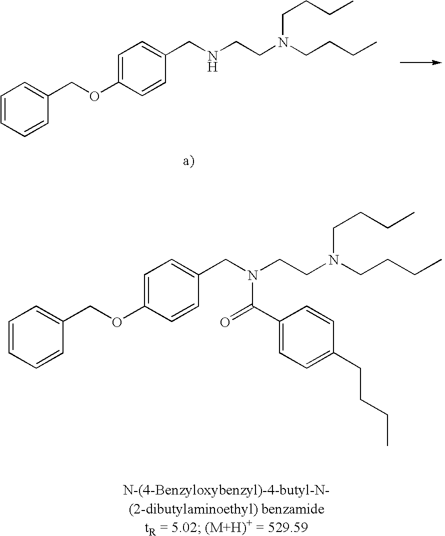

### Example 11

According to typical procedure B), the secondary amine b), obtained via typical procedure A), is reacted with 4-n-butylbenzoyl chloride to give

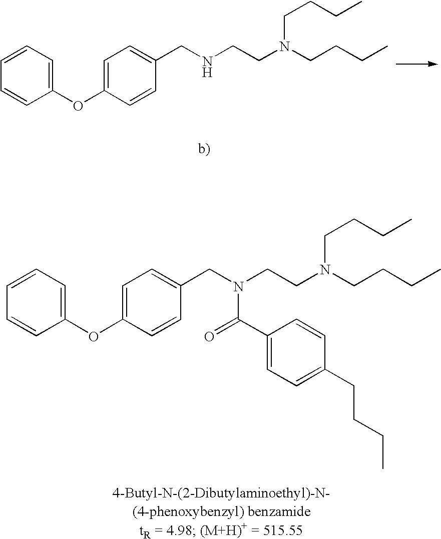

### Example 12

According to typical procedure B), the secondary amine c), obtained via typical procedure A), is reacted with 4-n-butylbenzoyl chloride to give

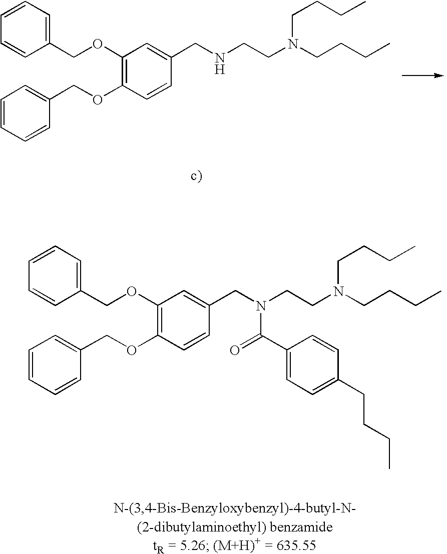

### Example 13

According to typical procedure B), the secondary amine d), obtained via typical procedure A), is reacted with 4-n-butylbenzoyl chloride to give

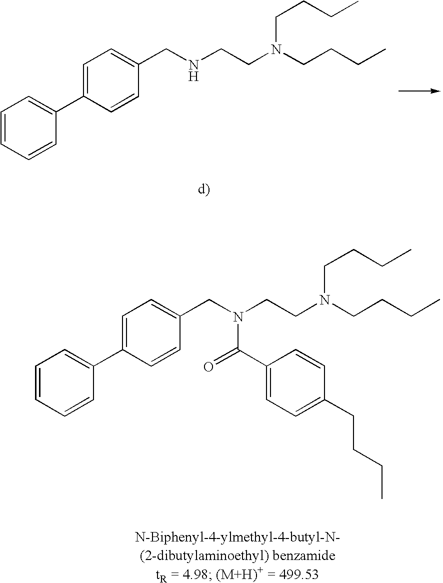

### Example 14

According to typical procedure B), the secondary amine e), obtained via typical procedure A), is reacted with 4-n-butylbenzoyl chloride to give

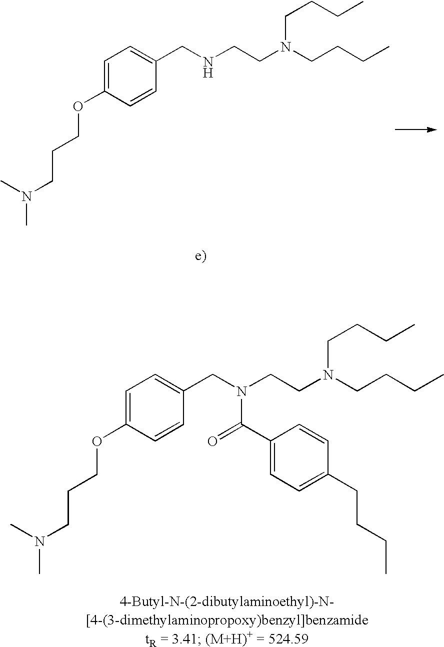

### Example 15

According to typical procedure B), the secondary amine f), obtained via typical procedure A), is reacted with 4-n-butylbenzoyl chloride to give

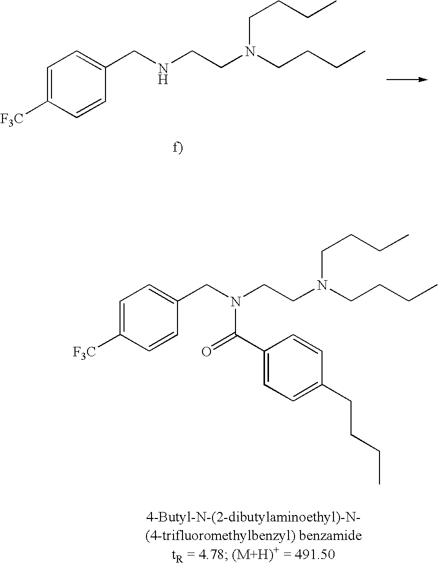

### Example 16

According to typical procedure B), the secondary amine g), obtained via typical procedure A), is reacted with 4-n-butylbenzoyl chloride to give

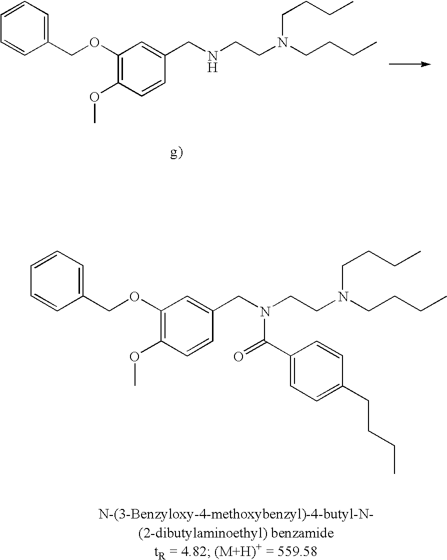

### Example 17

According to typical procedure B), the secondary amine g), obtained via typical procedure A), is reacted with 4-n-butylbenzoyl chloride to give

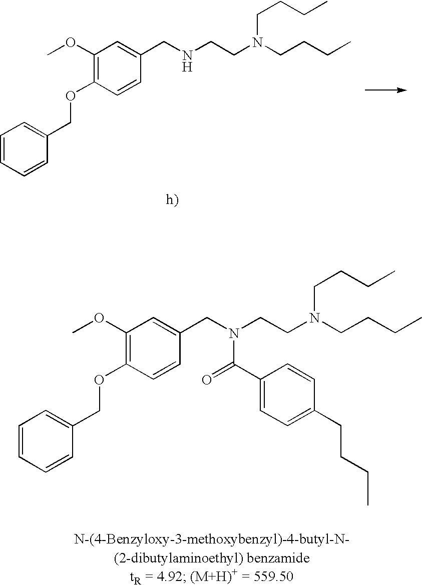

### Example 18

According to typical procedure B), the secondary amine a), obtained via typical procedure A), is reacted with 4-n-butylphenylisocyanate to give

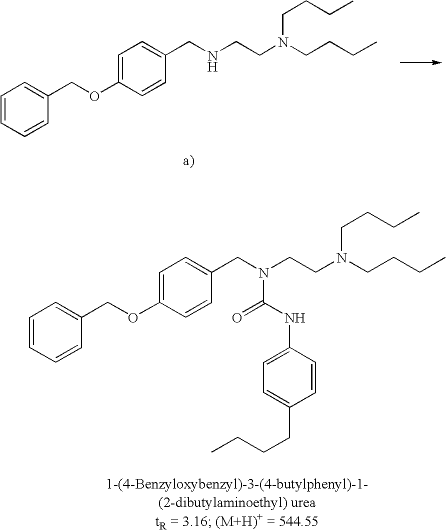

### Example 19

According to typical procedure B), the secondary amine e), obtained via typical procedure A), is reacted with4-n-butylphenylisocyanate to give

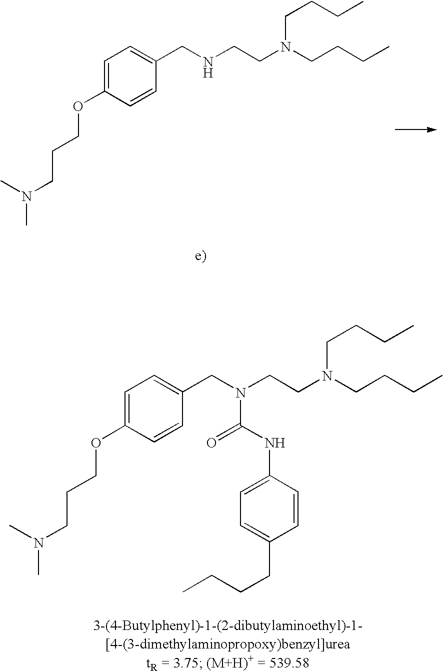

### Example 20

According to typical procedure C), the secondary amine a), obtained via typical procedure A), is reacted with 4-n-pentylbenzaldehyde to give

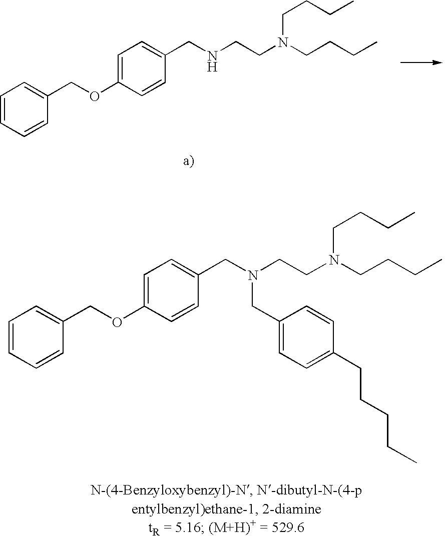

### Example 21

According to typical procedure B), the secondary amine b), obtained via typical procedure A), is reacted with 4-n-pentylbenzoyl chloride to give

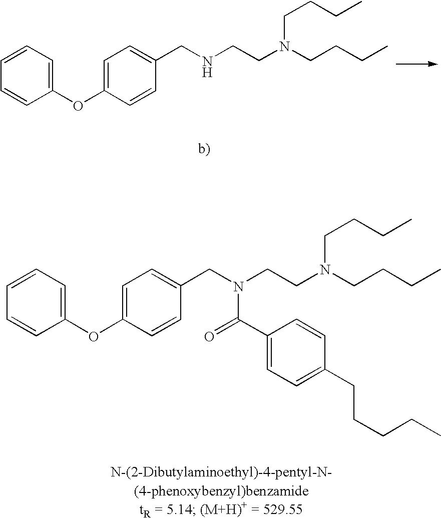

### Example 22

According to typical procedure B), the secondary amine c), obtained via typical procedure A), is reacted with 4-n-pentylbenzoyl chloride to give

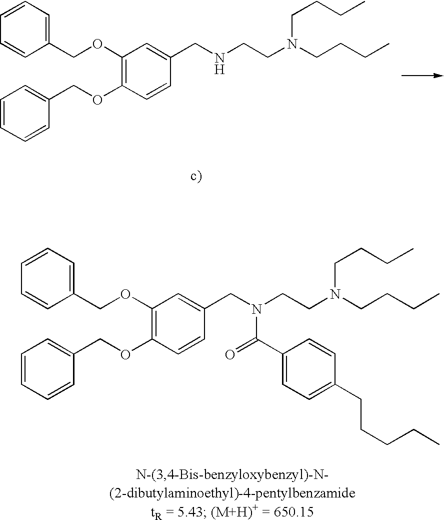

### Example 23

According to typical procedure B), the secondary amine d), obtained via typical procedure A), is reacted with 4-n-pentylbenzoyl chloride to give

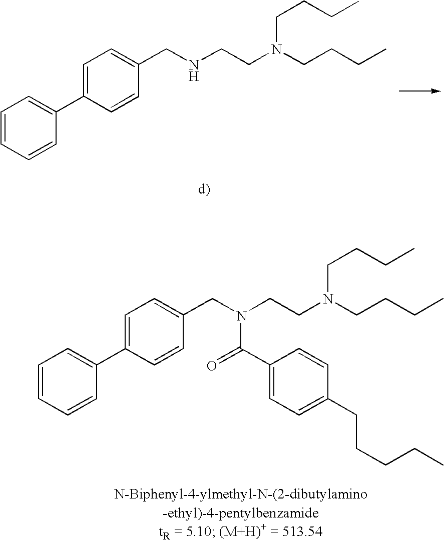

### Example 24

According to typical procedure B), the secondary amine e), obtained via typical procedure A), is reacted with 4-n-pentylbenzoyl chloride to give

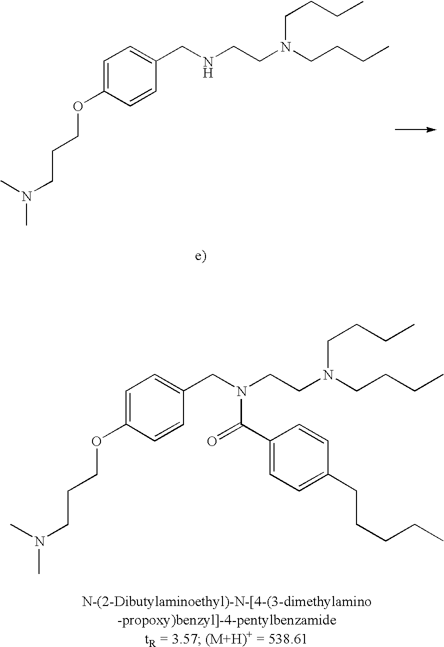

### Example 25

According to typical procedure B), the secondary amine f), obtained via typical procedure A), is reacted with 4-n-pentylbenzoyl chloride to give

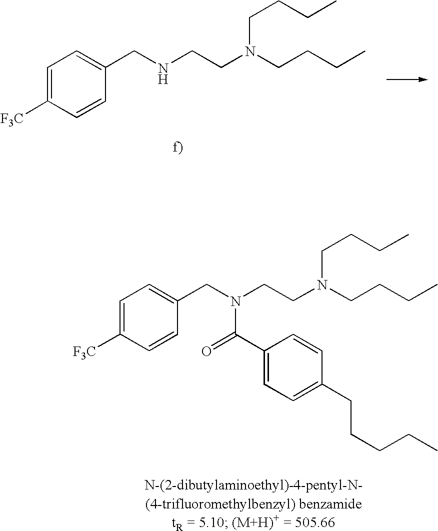

### Example 26

According to typical procedure B), the secondary amine g), obtained via typical procedure A), is reacted with 4-n-pentylbenzoyl chloride to give

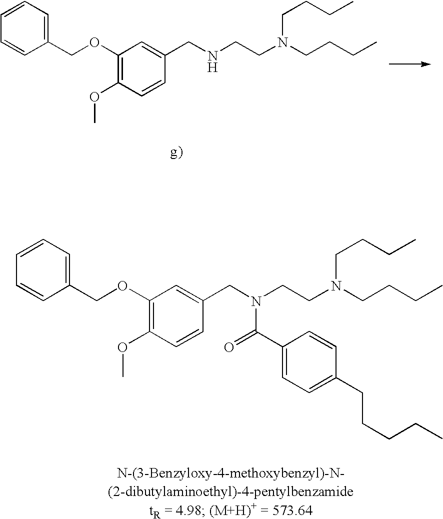

### Example 27

According to typical procedure B), the secondary amine h), obtained via typical procedure A), is reacted with 4-n-pentylbenzoyl chloride to give

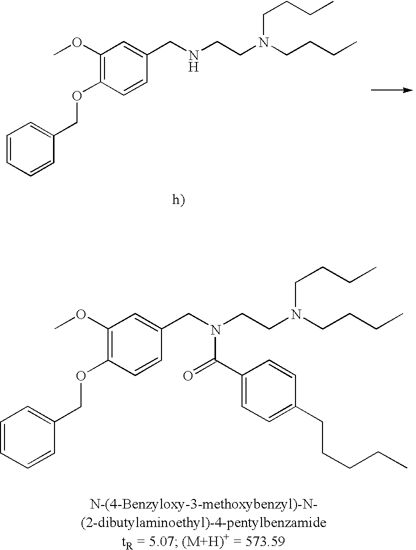

### Example 28

According to typical procedure B); the secondary amine a), obtained via typical procedure A), is reacted with 4-n-butoxybenzoyl chloride to give

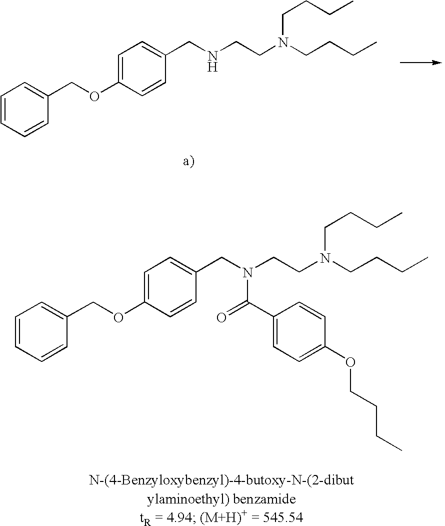

### Example 29

According to typical procedure B), the secondary amine b), obtained via typical procedure A), is reacted with 4-n-butoxybenzoyl chloride to give

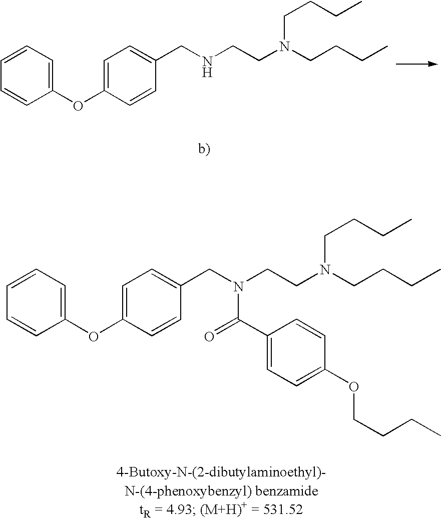

### Example 30

According to typical procedure B), the secondary amine c), obtained via typical procedure A), is reacted with 4-n-butoxybenzoyl chloride to give

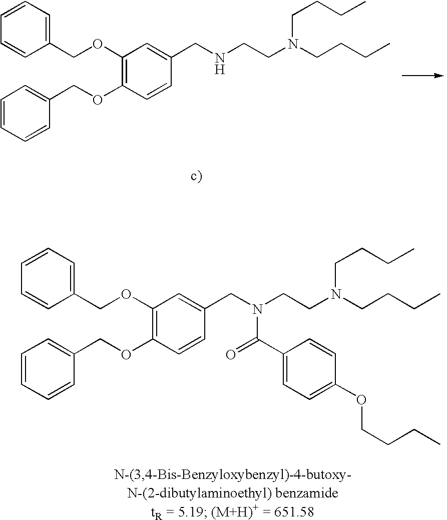

### Example 31

According to typical procedure B), the secondary amine d), obtained via typical procedure A), is reacted with 4-n-butoxybenzoyl chloride to give

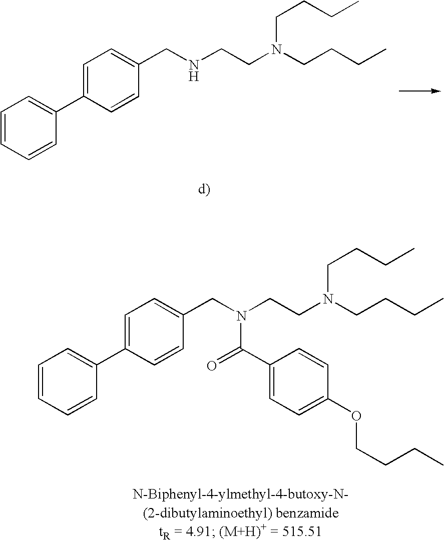

### Example 32

According to typical procedure B), the secondary amine e), obtained via typical procedure A), is reacted with 4-n-butoxybenzoyl chloride to give

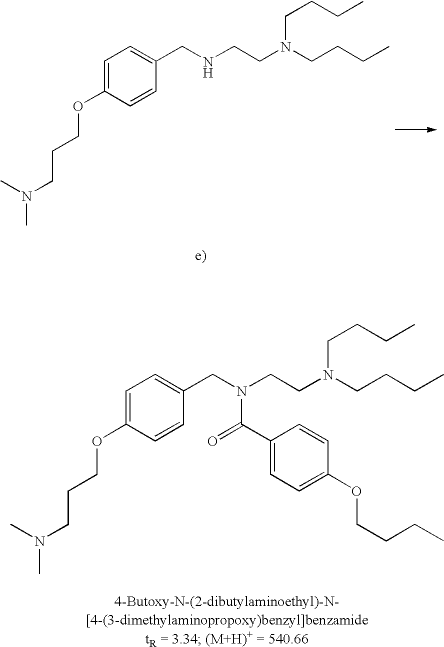

### Example 33

According to typical procedure B), the secondary amine f), obtained via typical procedure A), is reacted with 4-n-butoxybenzoyl chloride to give

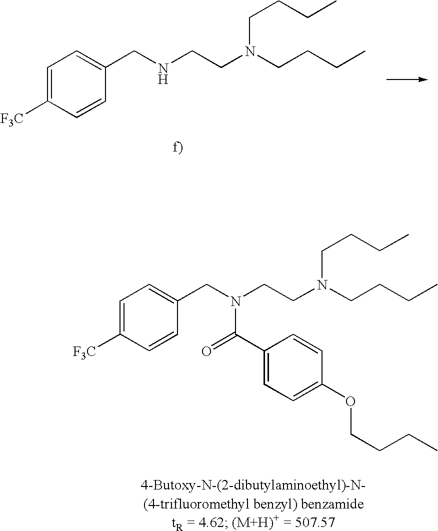

### Example 34

According to typical procedure B), the secondary amine g), obtained via typical procedure A), is reacted with 4-n-butoxybenzoyl chloride to give

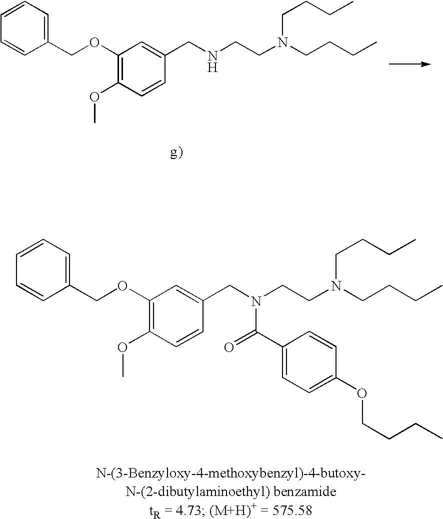

### Example 35

According to typical procedure B), the secondary amine h), obtained via typical procedure A), is reacted with 4-n-butoxybenzoyl chloride to give

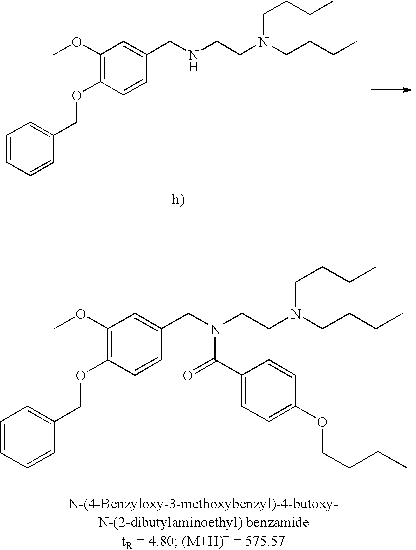

### Example 36

According to typical procedure B), the secondary amine i), obtained via typical procedure A), is reacted with 4-n-propylbenzoyl chloride to give

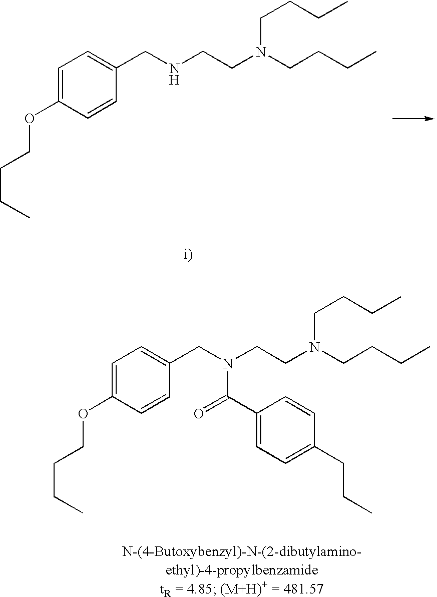

### Example 37

According to typical procedure B), the secondary amine i), obtained via typical procedure A), is reacted with 4-n-butylbenzoyl chloride to give

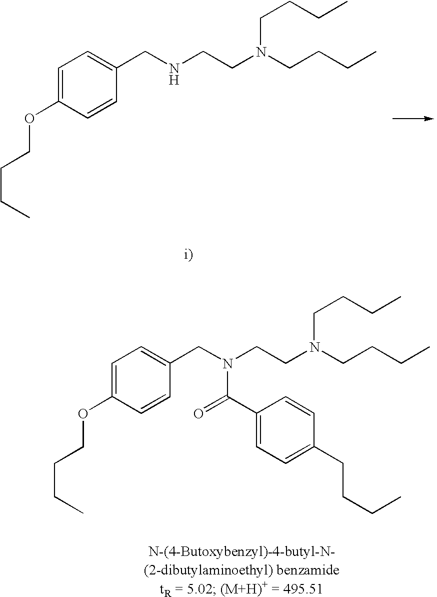

### Example 38

According to typical procedure B), the secondary amine i), obtained via typical procedure A), is reacted with 4-n-pentylbenzoyl chloride to give

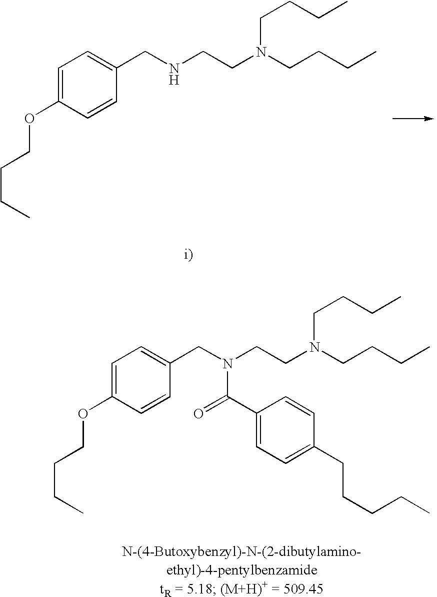

### Example 39

According to typical procedure B), the secondary amine D), obtained via typical procedure A), is reacted with 4-n-butoxybenzoyl chloride to give

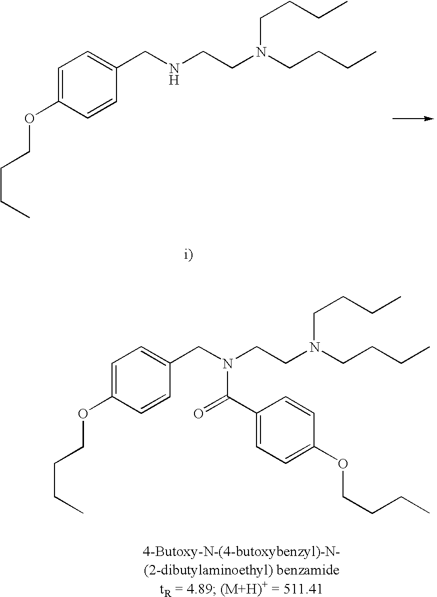

### Example 40

According to typical procedure B), the secondary amine b), obtained via typical procedure A), is reacted with 4-n-butylphenylisocyanate to give

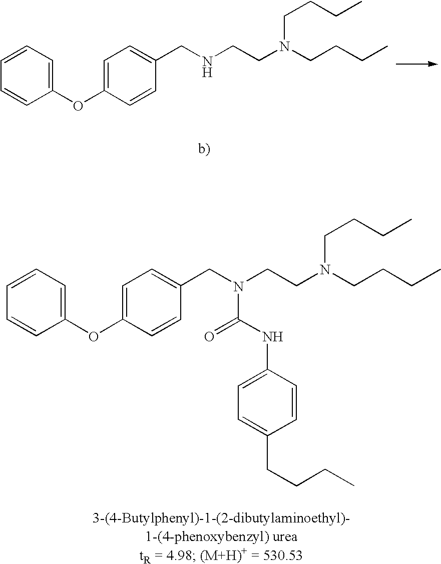

### Example 41

According to typical procedure B), the secondary amine c), obtained via typical procedure A), is reacted with 4-n-butylphenylisocyanate to give

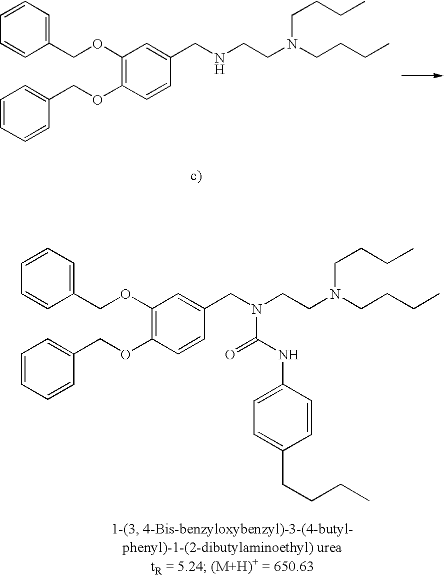

### Example 42

According to typical procedure B), the secondary amine d), obtained via typical procedure A), is reacted with 4-n-butylphenylisocyanate to give

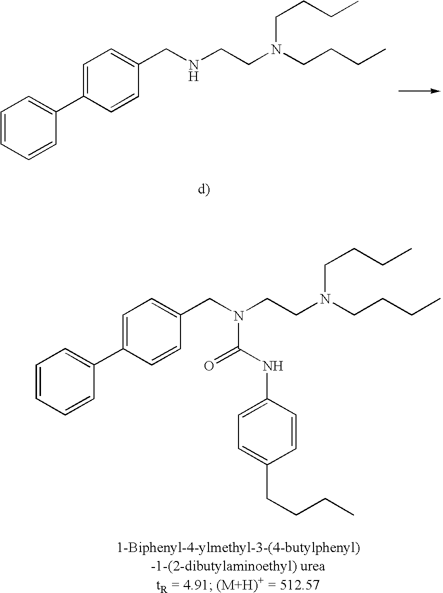

### Example 43

According to typical procedure B), the secondary amine i), obtained via typical procedure A), is reacted with 4-n-butylphenylisocyanate to give

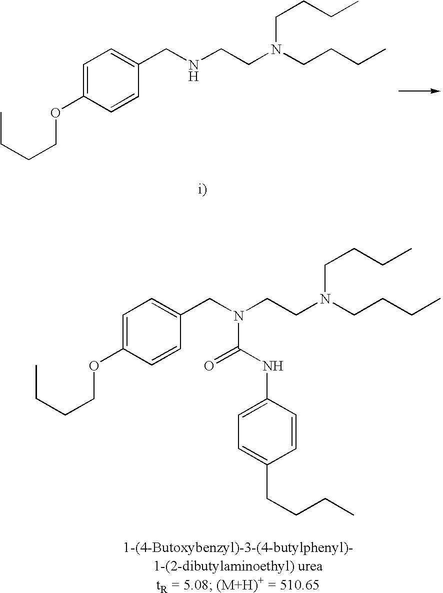

### Example 44

According to typical procedure B), the secondary amine f), obtained via typical procedure A), is reacted with 4-n-butylphenylisocyanate to give

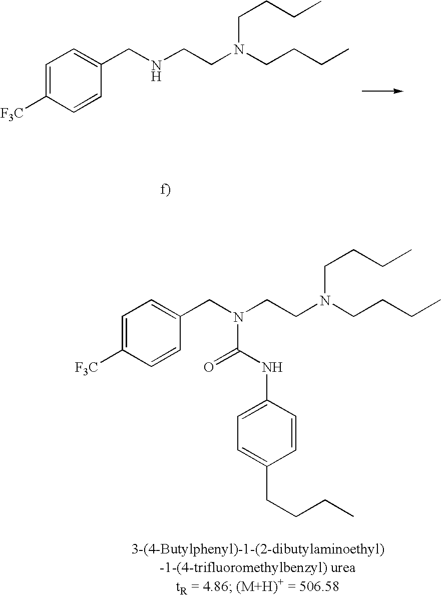

### Example 45

According to typical procedure B), the secondary amine g), obtained via typical procedure A), is reacted with 4-n-butylphenylisocyanate to give

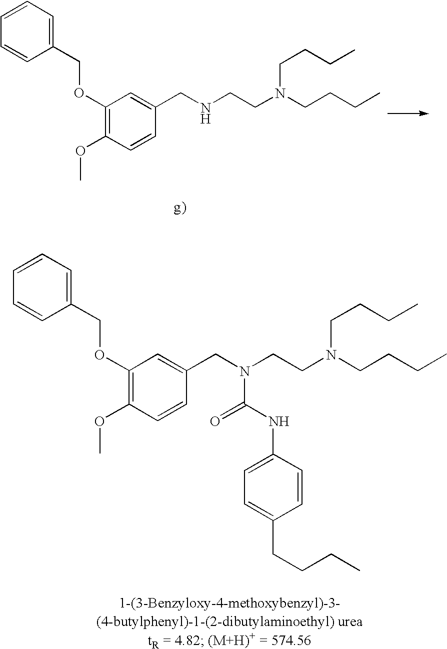

### Example 46

According to typical procedure B), the secondary amine h), obtained via typical procedure A), is reacted with 4-n-butylphenylisocyanate to give

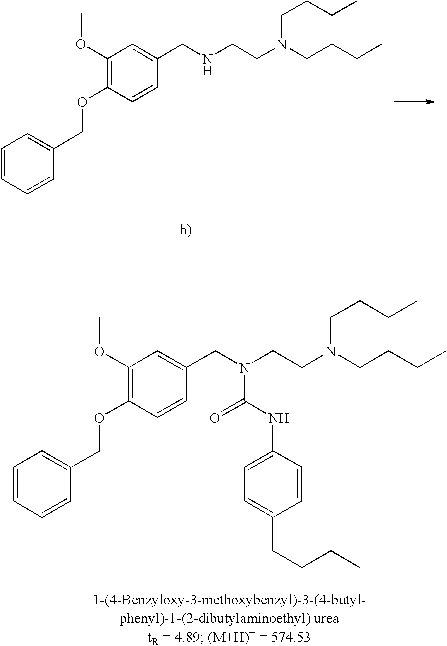

### Example 47

According to typical procedure B), the secondary amine k), obtained via typical procedure A), is reacted with 4-n-pentylbenzoyl chloride to give

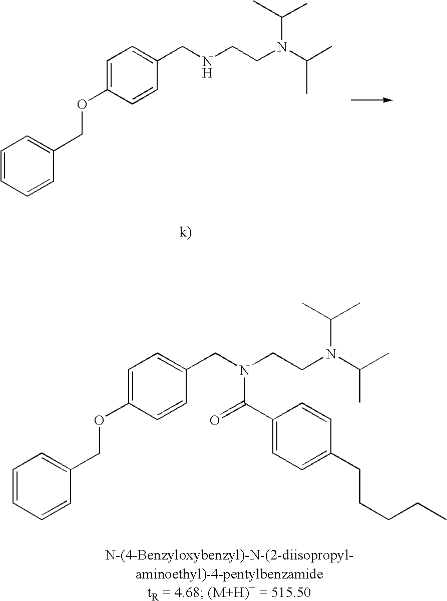

### Example 48

According to typical procedure B), the secondary amine I), obtained via typical procedure A), is reacted with 4-n-pentylbenzoyl chloride to give

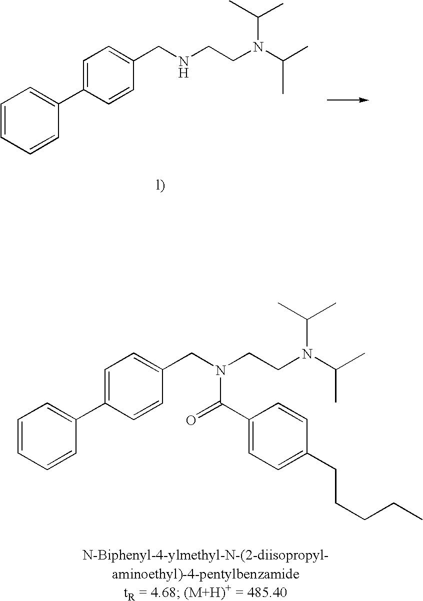

### Example 49

According to typical procedure B), the secondary amine m), obtained via typical procedure A), is reacted with 4-n-pentylbenzoyl chloride to give

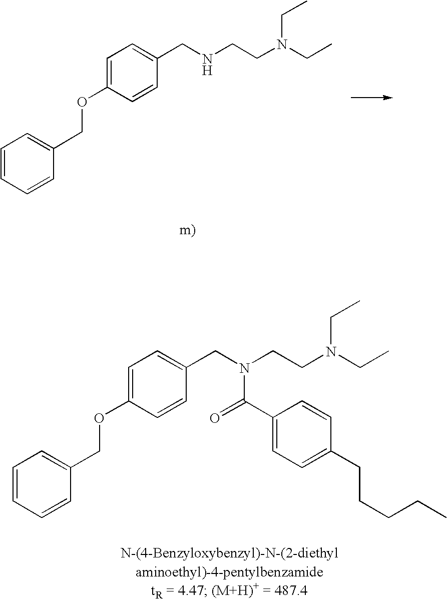

### Example 50

According to typical procedure B), the secondary amine n), obtained via typical procedure A), is reacted with 4-n-pentylbenzoyl chloride to give

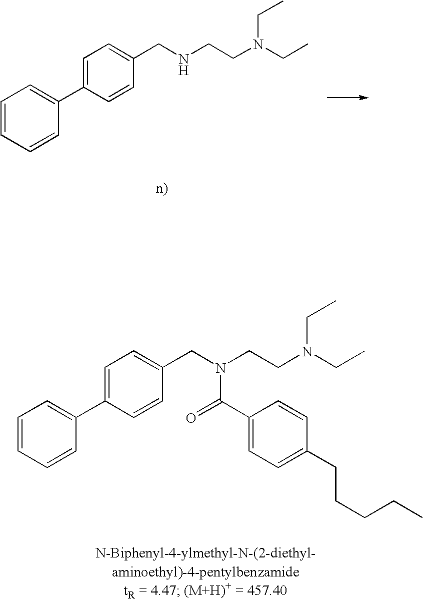

### Example 51

According to typical procedure B), the secondary amine o), obtained via typical procedure A), is reacted with 4-n-pentylbenzoyl chloride to give

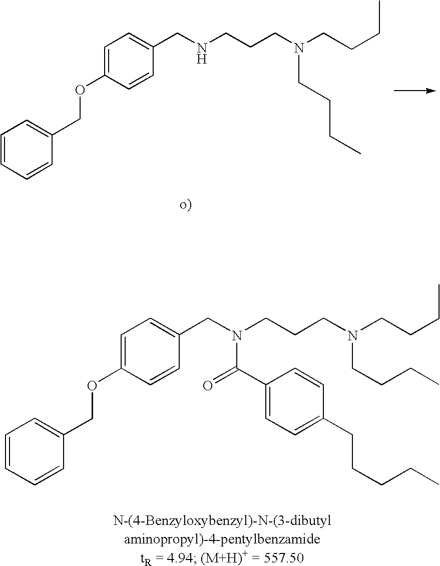

### Example 52

According to typical procedure B), the secondary amine p), obtained via typical procedure A), is reacted with 4n-pentylbenzoyl chloride to give

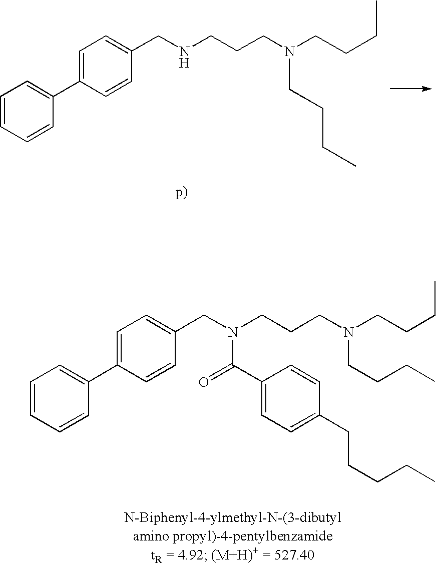

### Example 53

According to typical procedure B), the secondary amine q), obtained via typical procedure A), is reacted with 4-n-pentylbenzoyl chloride to give

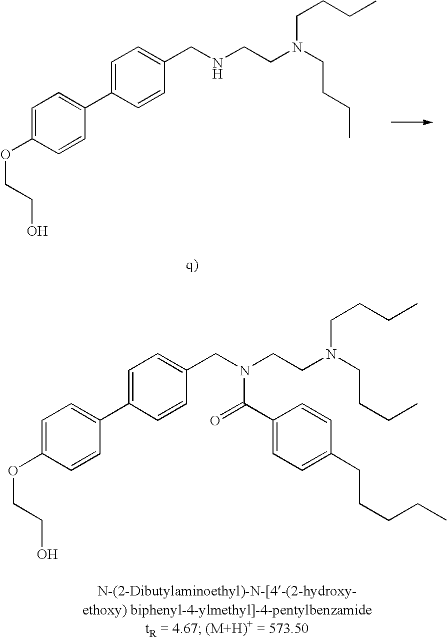

### Example 54

According to typical procedure B), the secondary amine q1), obtained via typical procedure A), is reacted with 4-n-pentylbenzoyl chloride to give

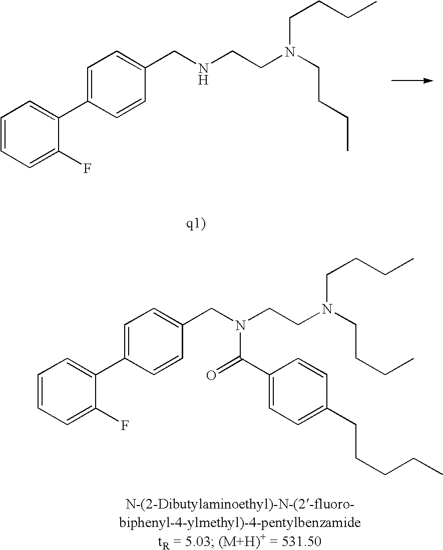

### Example 55

According to typical procedure B), the secondary amine q2), obtained via typical procedure A), is reacted with 4-n-pentylbenzoyl chloride to give

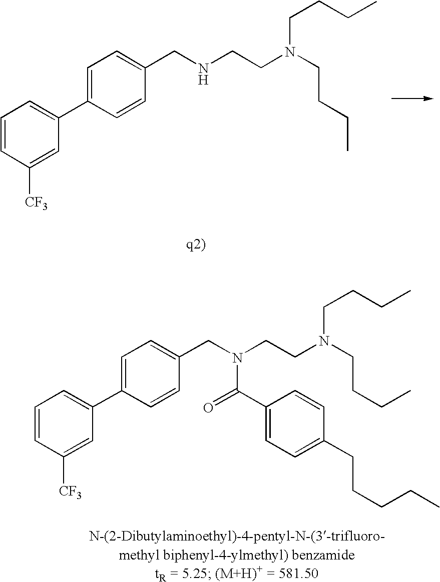

### Example 56

According to typical procedure B), the secondary amine q3), obtained via typical procedure A), is reacted with 4-n-pentylbenzoyl chloride to give

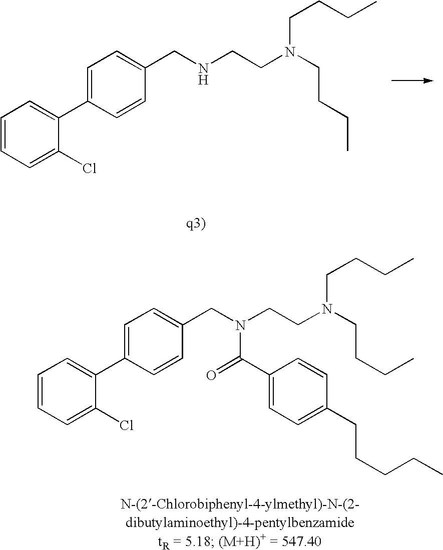

### Example 57

According to typical procedure B), the secondary amine q4), obtained via typical procedure A), is reacted with 4-n-pentylbenzoyl chloride to give

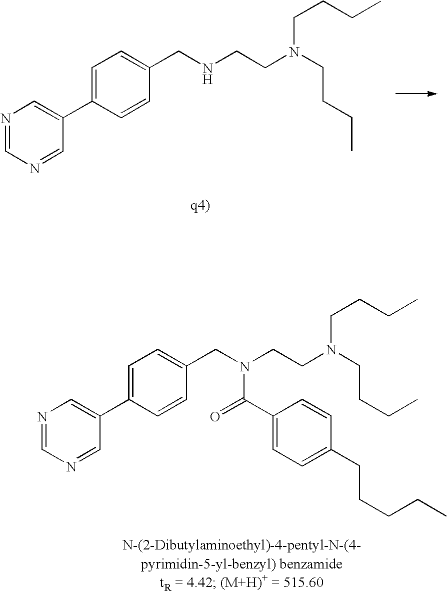

### Example 58

According to typical procedure B), the secondary amine q5), obtained via typical procedure A), is reacted with 4-n-pentylbenzoyl chloride to give

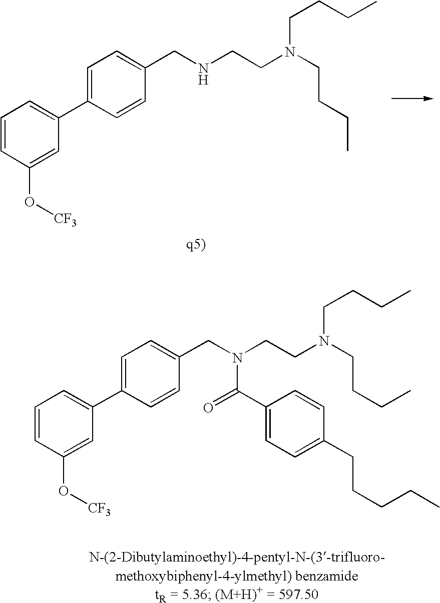

### Example 59

According to typical procedure B), the secondary amine q6), obtained via typical procedure A), is reacted with 4-n-pentylbenzoyl chloride to give

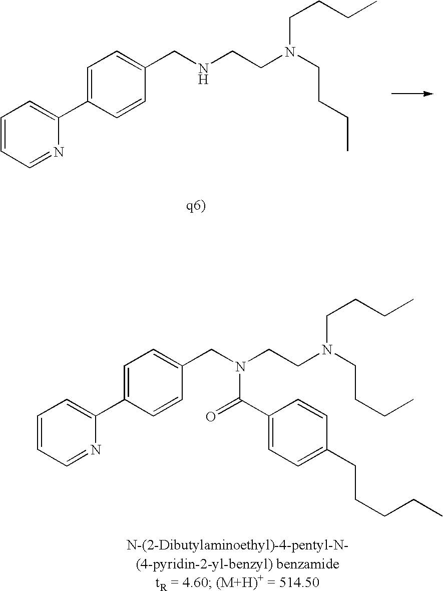

### Example 60

According to typical procedure B), the secondary amine q7), obtained via typical

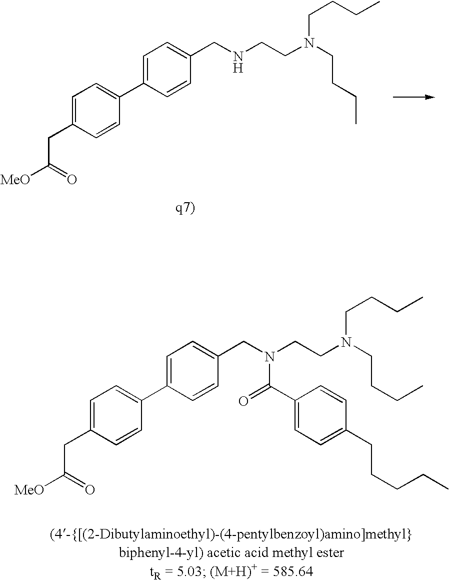

### Example 61

According to typical procedure B), the secondary amine q8), obtained via typical procedure A), is reacted with 4-n-pentylbenzoyl chloride to give

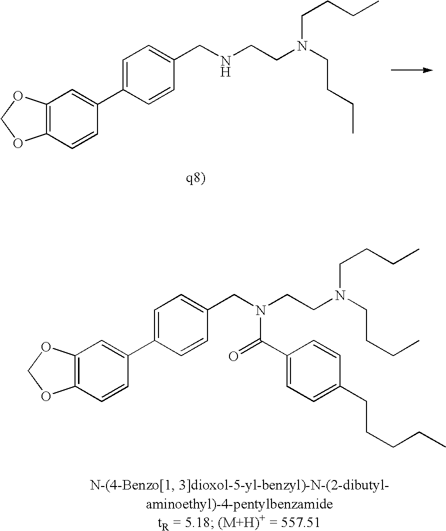

### Example 62

According to typical procedure B), the secondary amine q9), obtained via typical procedure A), is reacted with 4-n-pentylbenzoyl chloride to give

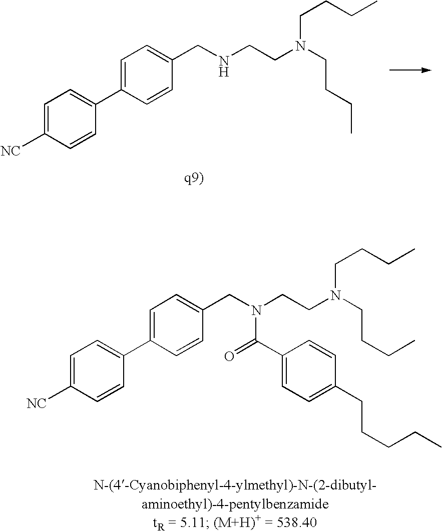

### Example 63

According to typical procedure B), the secondary amine q10), obtained via typical procedure A), is reacted with 4-n-pentylbenzoyl chloride to give

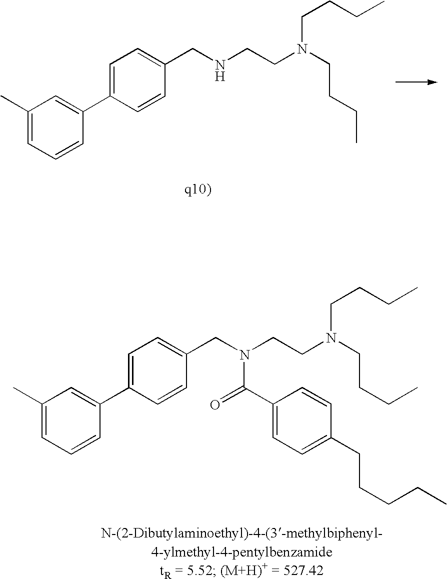

### Example 64

According to typical procedure C), the secondary amine q2), obtained via typical procedure A), is reacted with 4-n-pentylbenzaldehyde to give

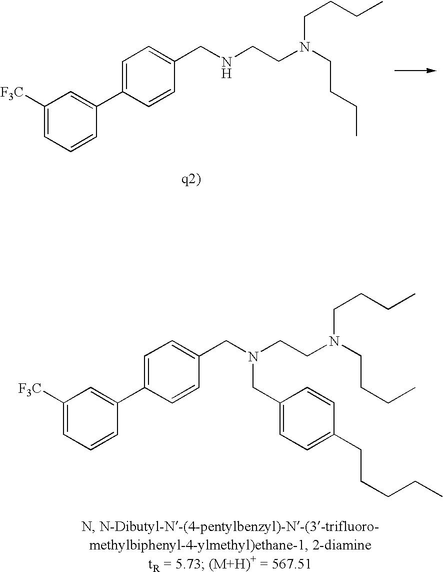

### Example 65

According to typical procedure C), the secondary amine q), obtained via typical procedure A), is reacted with 4-n-pentylbenzaldehyde to give

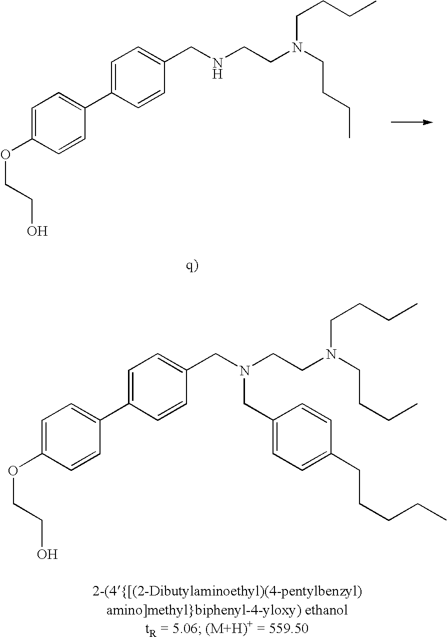

### Example 66

According to typical procedure C), the secondary amine q8), obtained via typical procedure A), is reacted with 4-n-pentylbenzaldehyde to give

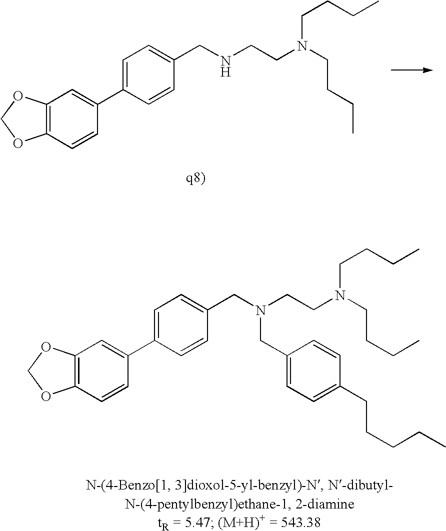

### Example 67

According to typical procedure C), the secondary amine q10), obtained via typical procedure A), is reacted with 4-n-pentylbenzaldehyde to give

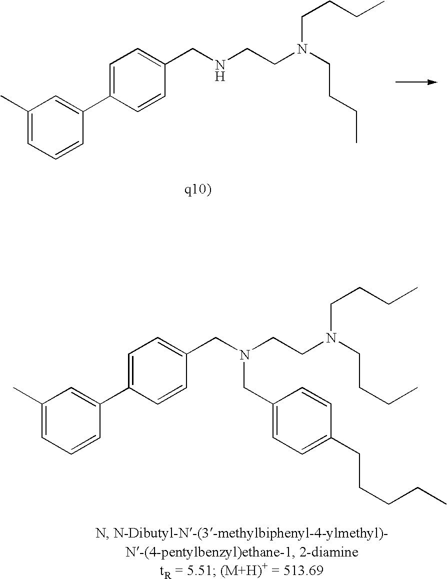

### Example 68

According to typical procedure C), the secondary amine q9), obtained via typical procedure A), is reacted with 4-n-pentylbenzaldehyde to give

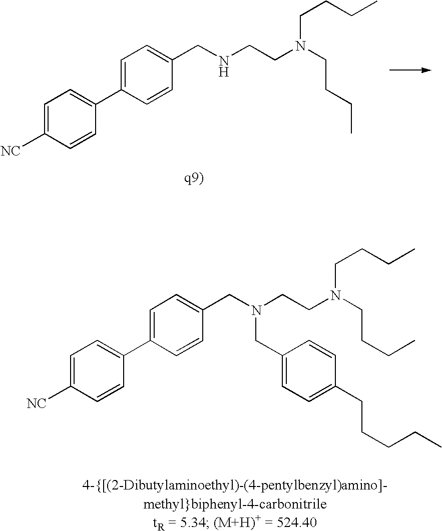

### Example 69

According to typical procedure B), the secondary amine r), obtained via typical procedure A), is reacted with 4-n-pentylbenzoyl chloride to give

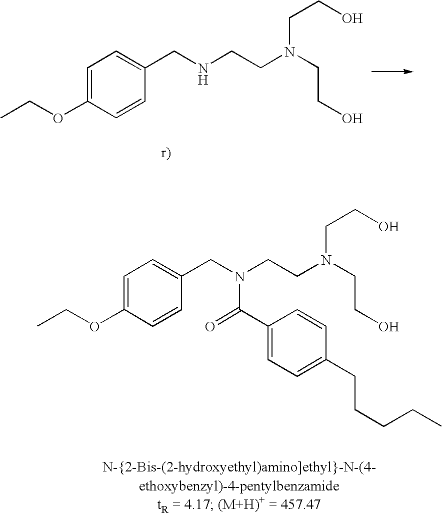

### Example 70

According to typical procedure B), the secondary amine s), obtained via typical procedure A), is reacted with 4-n-pentylbenzoyl chloride to give

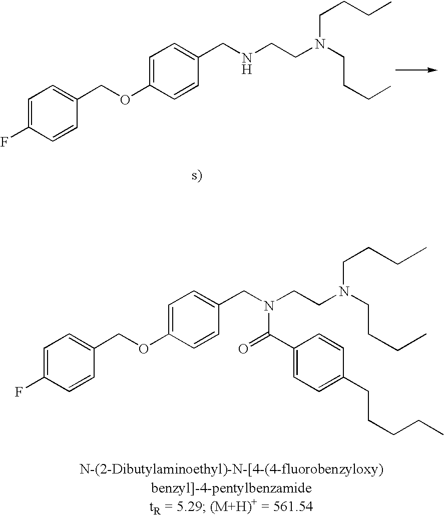

### Example 71

According to typical procedure B), the secondary amine t), obtained via typical procedure A), is reacted with 4-n-pentylbenzoyl chloride to give

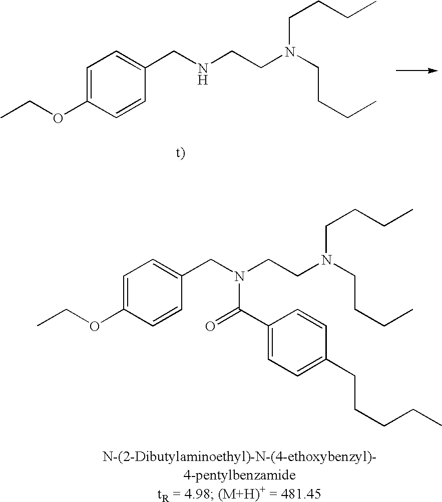

### Example 72

According to typical procedure B), the secondary amine u), obtained via typical procedure A), is reacted with 4-n-pentylbenzoyl chloride to give

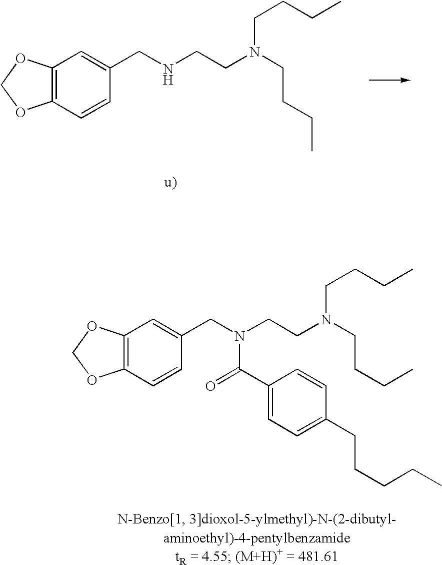

### Example 73

According to typical procedure C), the secondary amine a), obtained via typical procedure A), is reacted with pyridine-2-carbaldehyde to give

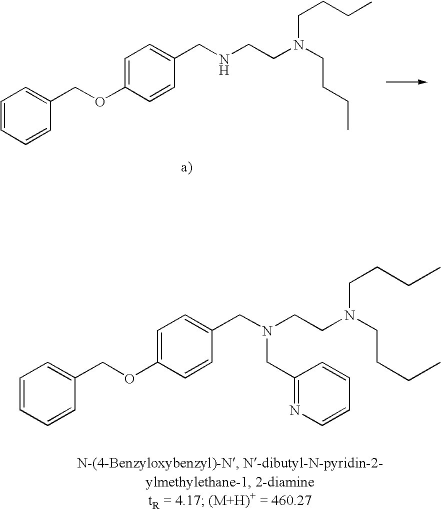

### c) Referential Examples

### Referential Example 1

According to typical procedure D), 4-formylbenzeneboronic acid is coupled with 2-(4-bromophenoxy)ethanol to give

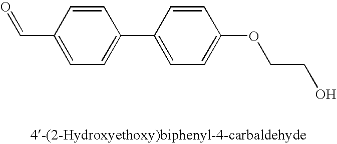

### Referential Example 2

According to typical procedure D), 4-formylbenzeneboronic acid is coupled with 1-bromo-2-fluorobenzene to give

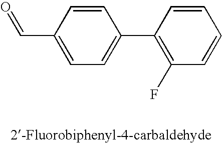

### Referential Example 3

According to typical procedure D), 4-formylbenzeneboronic acid is coupled with 3-bromobenzotrifluoride to give

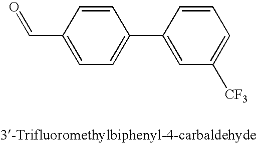

### Referential Example 4

According to typical procedure D), 4-formylbenzeneboronic acid is coupled with 1-bromo-2-chlorobenzene to give

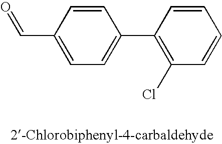

### Referential Example 5

According to typical procedure D), 4-formylbenzeneboronic acid is coupled with 5-bromopyrimidine to give

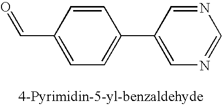

### Referential Example 6

According to typical procedure D), 4-formylbenzeneboronic acid is coupled with 1-bromo-3-(trifluoromethoxy)benzene to give

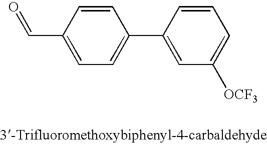

### Referential Example 7

According to typical procedure D), 4-formylbenzeneboronic acid is coupled with 5-bromobenzo[1,3]dioxole to give

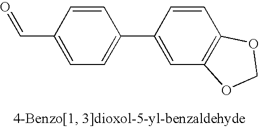

### Referential Example 8

According to typical procedure D), 4-formylbenzeneboronic acid is coupled with 4-bromobenzonitrile to give

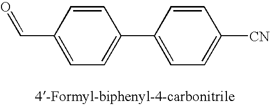

### Referential Example 9

According to typical procedure D), 4-formylbenzeneboronic acid is coupled with 3-bromotoluene to give

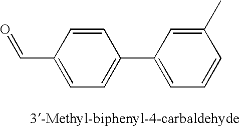

### Referential Example 10

According to typical procedure D), 4-formylbenzeneboronic acid is coupled with 4-bromophenyl acetic acid methyl ester to give

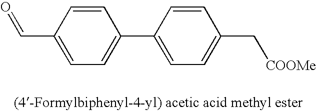

1. Compounds of the general formula I 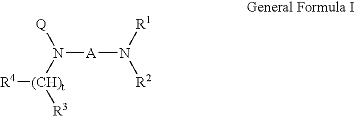
wherein
Q represents —SO₂—R⁵; —CO—R⁵; —CO—NH—R⁵; —CO—N(R⁵)(R⁶); —CO—OR⁵; —(CH₂)ₚ—R⁵; —(CH₂)ₚ—CH(R⁵)(R⁶);
R¹ and R² represent propyl; butyl; pentyl; hexyl; ω-hydroxy-propyl; ω-hydroxy-butyl; ω-hydroxy-pentyl; ω-hydroxy-hexyl; lower alkoxy-propyl; lower alkoxy-butyl; lower alkoxy-pentyl; lower alkoxy-hexyl; aryl-lower alkyl; cycloalkyl; cycloalkyl-lower alkyl; heterocyclyl; and can be the same or different; or R¹ and R² and the nitrogen atom together can represent a ring such as azetidin; azepan;
R³ represents lower alkyl; lower alkenyl; aryl; heteroaryl; cycloalkyl; heterocyclyl; aryl-lower alkyl; heteroaryl-lower alkyl; cycloalkyl-lower alkyl; heterocyclyl-lower alkyl; aryl-lower alkenyl; heteroaryl-lower alkenyl; cycloalkyl-lower alkenyl; heterocyclyl-lower alkenyl;
R⁴ represents hydrogen; —CH₂—OR⁷; —CO—OR⁷; lower alkyl;
R⁵ and R⁶ represent lower alkyl; lower alkenyl; aryl; heteroaryl; cycloalkyl; heterocyclyl; aryl-lower alkyl; heteroaryl-lower alkyl; cycloalkyl-lower alkyl; heterocyclyl-lower alkyl; aryl-lower alkenyl; heteroaryl-lower alkenyl; cycloalkyl-lower alkenyl; heterocyclyl-lower alkenyl;
R⁷ represents hydrogen, lower alkyl; cycloalkyl; aryl; cycloalkyl-lower alkyl; aryl-lower alkyl;
t represents the whole numbers 0 (zero) or 1 and in case t represents the whole number 0 (zero), R⁴ is absent;
p represents the whole numbers 0 (zero), 1 or 2;
A represents —(CH₂)ₙ—;
n represents the whole numbers 2, 3, 4 or 5;
and pure enantiomers, mixtures of enantiomers, pure diastereomers, mixtures of diastereomers, diastereomeric racemates, mixtures of diastereomeric racemates and pharmaceutically acceptable salts thereof

2. Compounds of formula II 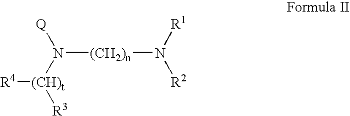
wherein
Q, t, R³ and R⁴ are as defined in general formula I above, R¹ and R² represent lower alkyl and n represents the whole numbers 2 or 3
and pure enantiomers, mixtures of enantiomers, pure diastereomers, mixtures of diastereomers, diastereomeric racemates, mixtures of diastereomeric racemates and pharmaceutically acceptable salts thereof.

3. Compounds of formula III 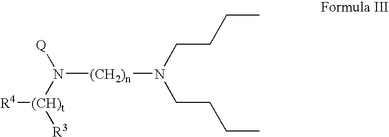
wherein
Q, t, R³ and R⁴ are as defined in general formula I above and n represents the whole numbers 2 or 3
and pure enantiomers, mixtures of enantiomers, pure diastereomers, mixtures of diastereomers, diastereomeric racemates, mixtures of diastereomeric racemates and pharmaceutically acceptable salts thereof.

4. Compounds of formula IV 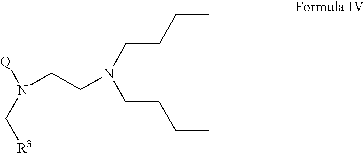
wherein
Q and R³ are as defined in general formula I above
and pure enantiomers, mixtures of enantiomers, pure diastereomers, mixtures of diastereomers, diastereomeric racemates, mixtures of diastereomeric racemates and pharmaceutically acceptable salts thereof.

5. Compounds of formula V 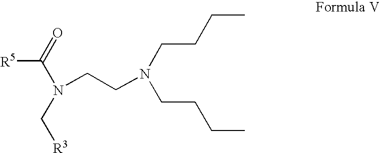
wherein R³ and R⁵ are as defined in general formula I above
and pure enantiomers, mixtures of enantiomers, pure diastereomers, mixtures of diastereomers, diastereomeric racemates, mixtures of diastereomeric racemates and pharmaceutically acceptable salts thereof.

6. Compounds of formula VI 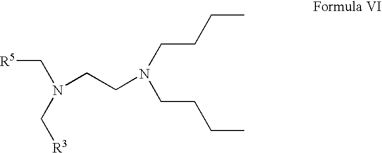
wherein R³ and R⁵ are as defined in general formula I above
and pure enantiomers, mixtures of enantiomers, pure diastereomers, mixtures of diastereomers, diastereomeric racemates, mixtures of diastereomeric racemates and pharmaceutically acceptable salts thereof.

7. The compounds according to any one of claims 1-6
N-(4-Benzyloxybenzyl)-N-(2-dibutylamino-ethyl)-4-pentylbenzamide;
N-Biphenyl-4-ylmethyl-N-(2-dibutylamino-ethyl)-4-pentylbenzamide;
N-(2-Dibutylaminoethyl)-N-[4′-(2-hydroxy-ethoxy)-biphenyl-4-ylmethyl]-4-pentylbenzamide;
N-(4-Benzo[1,3]dioxol-5-yl-benzyl)-N-(2-dibutyl-aminoethyl)-4-pentylbenzamide.

8. Pharmaceutical compositions containing one or more compounds as claimed in any one of claims 1 to 7 and inert excipients.

9. Pharmaceutical compositions according to claim 8 for treatment of diseases demanding the inhibition of aspartic proteases.

10. Pharmaceutical compositions according to claim 8 for treatment of disorders associated with the role of plasmepsin II and which require selective inhibition of plasmepsin II.

11. Pharmaceutical compositions according to claim 8 for treatment or prevention of malaria.

12. Pharmaceutical compositions according to claim 8, which contain aside of one or more compounds of the general formula I a known inhibitor of plasmepsin II, HIV protease or cathepsin D or E.

13. A process for the preparation of a pharmaceutical composition according to any one of claims 9 to 12, characterized by mixing one or more active ingredients according to any one of claims 1 to 7 with inert excipients in a manner known per se.

14. Use of at least one of the compounds of the general formula I for the treatment or prevention of diseases.

15. The novel compounds, processes and methods as well as the use of such compounds substantially as described herein before.
